(19) United States
(12) Patent Application Publication
(10) Pub. No.: US 2009/0175889 A1
(43) Pub. Date: Jul. 9, 2009

(54) HIV VACCINE

(75) Inventors: Shiu-Lok Hu, Redmond, WA (US); Yun Li, Bellevue, WA (US); Bradley Cleveland, Seattle, WA (US)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(73) Assignee: University of Washington, Seattle, WA (US)

(21) Appl. No.: 12/201,014

(22) Filed: Aug. 29, 2008

Related U.S. Application Data
(60) Provisional application No. 60/969,380, filed on Aug. 31, 2007.

(30) Foreign Application Priority Data
Aug. 31, 2007 (CA) 2597151

(51) Int. Cl. A61K 39/21; C12N 15/11; C12N 7/00; C07K 14/00; C12N 5/06; A61K 31/7088

(52) U.S. Cl. 424/184.1; 536/23.72; 435/235.1; 530/350; 435/375; 514/44

(57) ABSTRACT

The invention provides a polynucleotide encoding an envelope protein comprising gp120 of human immunodeficiency virus-1 (HIV-1) having a mutation at amino acid residues 197-199 whereby a single N-linked glycan is removed. In a typical embodiment, the mutation replaces the asparagine (N) at residue 197 with another amino acid, such as glutamine (Q), creating an N197Q mutation. Because the N197 glycan is highly conserved among HIV-1 subtypes, this approach is applicable across HIV-1 isolates. The invention provides polypeptides, polynucleotides encoding the polypeptides, vectors, and recombinant viruses containing the polynucleotides, antigen-presenting cells (APCs) presenting the polypeptides, immune cells directed against HIV, and pharmaceutical compositions. The invention additionally provides methods, including methods for preventing and treating infection, for killing infected cells, for inhibiting viral replication, for enhancing secretion of antiviral and/or immunomodulatory lymphokines, and for enhancing production of neutralizing antibody.

This application claims benefit of U.S. provisional patent application No. 60/969,380, filed Aug. 31, 2007, the entire contents of which are incorporated by reference into this application.

### STATEMENT REGARDING FEDERALLY SPONSORED RESEARCH

This invention was made with government support under grant number R21 AI042720 and 5P01 AI054564 awarded by the National Institutes of Health (NIH). The government has certain rights in the invention.

### TECHNICAL FIELD OF THE INVENTION

The invention relates to molecules, compositions and methods that can be used for the treatment and prevention of infection relating to the human immunodeficiency virus (HIV) and acquired immunodeficiency syndrome (AIDS). More particularly, the invention identifies a single N-linked glycan in HIV-1 gp120 whose removal results in increased sensitivity to broadly neutralizing monoclonal antibodies and the ability to mediate CD4-independent viral infection. Immunization with HIV-1 Env immunogens containing this modified HIV-1 gp120 results in enhanced neutralizing antibody responses, not only against the mutant gp120 itself, but also against the homologous wild type virus and a panel of heterologous primary subtype B HIV-1 isolates. In addition, the invention relates to methods for preventing and treating HIV infection and influencing related immune responses.

### BACKGROUND OF THE INVENTION

Induction of broadly neutralizing antibodies (NtAb) against primary isolates of human immunodeficiency virus (HIV) remains a major and unmet goal for AIDS vaccine research. Early attempts using envelope-based vaccines have elicited antibodies that are effective only against laboratory-adapted isolates. In these instances, protection has been correlated with high-titered NtAb directed to the V3 hypervariable region of gp120. However, neutralizing activities generated are largely isolate specific and are minimally effective against most primary isolates of HIV-1. The failure of subunit gp120 vaccines to protect against HIV-1 acquisition in Phase III clinical trials underscores the difficulty of the task.

Nevertheless, NtAb can often be found in HIV infected individuals. Responses generated early in infection are usually narrow in specificity, neutralizing the transmitted viruses in the host, but not the contemporaneous ones. Such responses broaden during the course of infection in some long-term survivors who are able to control their infection in the absence of antiviral treatment. However, the nature of the cross-neutralizing antibody response and the mechanisms leading to its genesis are not well understood.

The envelope antigens of HIV-1, like those of many other lentiviruses, are extensively glycosylated. The surface antigen gp120 contains both N-linked and O-linked glycans, contributing to nearly 50% of its molecular mass. These carbohydrates play an important role in the structure and function of the envelope glycoproteins, including virus assembly, receptor binding, and syncytia formation. In addition, multiple studies have shown that carbohydrate moieties on viral envelope modulate its antigenicity and the sensitivity of the virus to neutralizing antibodies. However, such effect could be enhancing or interfering, depending on the specific combination of antibody and glycan involved.

Despite considerable evidence indicating the role of glycosylation in modulating Env antigenicity, relatively few have addressed its potential role in influencing the immunogenicity of HIV-1 envelope proteins. Haigwood et al. (1992) compared immune responses generated by gp120 produced in mammalian (native and glycosylated) versus those produced in yeast cells (denatured and non-glycosylated) and found that the native structure is superior in inducing broad spectrum neutralizing antibodies in baboons. Benjouad et al. (1992) analyzed the specificity and neutralizing capacity of antibodies raised against native or deglycosylated forms of gp120 generated by various enzymatic reactions. Both investigators compared immunogens that have global differences in their glycosylation patterns and perhaps overall structure. We and others have examined effects of site-specific deglycosylations, but have not observed any major difference between immune responses elicited by wild-type (WT) versus modified envelope proteins (Bolmstedt et al., 1996; Bolmstedt et al., 2001; Quinones-Kochs et al., 2002; Burke et al., 2006). On the other hand, Desrosiers and colleagues (Reitter and Desrosiers, 1998; Reitter et al., 1998) reported significant increase in antigenicity and immunogenicity of mutant forms of SIV depleted of N-linked glycans in the V1 region of its envelope proteins. Immunization with a multiply deglycosylated SIVmac239 Env, however, failed to protect against homologous virus challenge (Mori et al., 2005).

### SUMMARY OF THE INVENTION

The invention provides a polynucleotide encoding an envelope protein comprising gp120 of human immunodeficiency virus-1 (HIV-1). Also provided are HIV-1 Env immunogens containing the gp120 protein. The gp120 has a mutation at amino acid residues 197-199 that effects removal of a single N-linked glycan relative to wild type gp120. In a typical embodiment, the mutation replaces the asparagine (N) at residue 197 with another amino acid. The substituted amino acid can be glutamine (Q), creating an N197Q mutation. One example of a polynucleotide encoding a gp120 envelope protein having the N197Q mutation is described by SEQ ID NO: 1. Other amino acids, such as alanine (A) can be substituted for the asparagine as well, and still remove the N-linked glycan. Alternatively, one can modify the T or S at residue 199 to remove the glycosylation site. Because the N197 glycan is highly conserved among all HIV-1 subtypes, this approach is applicable across HIV-1 isolates. The polynucleotide or protein can be provided in the form of a pharmaceutical composition together with a pharmaceutically acceptable carrier and, optionally, an adjuvant.

In a typical embodiment, the envelope protein comprises gp140 or gp160. Including at least the ectodomain of gp41 (gp41e) helps the gp120 maintain its trimeric structure. Alternatively, one skilled in the art is aware of other means to enhance the form and stability of the gp120 protein.

Also provided is a pharmaceutical composition comprising a recombinant virus genetically modified to express an envelope protein comprising gp120 of human immunodeficiency virus-1 (HIV-1), wherein the gp120 has a mutation at amino acid residues 197-199, and wherein the mutation effects removal of a single N-linked glycan relative to wild type gp120. In one embodiment, the virus is an adenovirus, adeno-associated virus, pox virus, or alphavirus. One example of a pox virus is a vaccinia virus. In another embodiment, the recombinant virus is an attenuated HIV virus.

The invention further provides a method of producing immune cells directed against HIV comprising contacting an immune cell with an antigen-presenting cell, wherein the antigen-presenting cell is modified to present an envelope protein comprising gp120 of human immunodeficiency virus-1 (HIV-1), or portions thereof. The gp120 has a mutation at amino acid residues 197-199 whereby a single N-linked glycan is removed. Immune cells produced by this method include antigen-specific B cells as well as T cells. The invention also provides polyclonal neutralizing antibodies elicited by immunization with the mutant gp120 of the invention as well as hybridomas producing monoclonal antibodies of desired specificity. Such antibodies can be used in passive immunization, for example, to prevent neonatal infection, or treatment of infected individuals. Similarly, the invention provides a method of enhancing production of HIV-neutralizing antibody in a subject. The method comprises contacting an HIV-infected cell in a subject with a composition of the invention.

Additional methods provided by the invention include a method of reducing plasma levels of HIV in a subject. The method comprises immunizing the subject with a composition of the invention. Also included are a method of maintaining CD4 cells in an HIV-infected subject, a method of prolonging disease-free survival in an HIV-infected subject, a method of inducing an immune response to an HIV-infection in a subject and a method of treating an HIV infection in a subject. The methods comprise administering a composition of the invention to the subject. In some embodiments, the method further comprises administering the composition a second time after at least 3 weeks following the previous administration. Those skilled in the art understand that an appropriate schedule for dosing and boosting will depend on the particular composition, mode of delivery and the subject's condition, including body weight. In many cases, for example, a boosting administration will occur 4-6 months following initial administration. For newborns, however, the schedule may be compressed to account for both the smaller body size and the underdeveloped immune system. One example of a prime-boost protocol includes priming with a recombinant virus, e.g., vaccinia modified to deliver the polynucleotide encoding a mutant gp120 of the invention, and boosting with the protein, e.g., the mutant gp120 of the invention.

### BRIEF DESCRIPTION OF THE DRAWINGS

FIG. 1: N-linked glycan mutants in HIV-1 Env. Potential N-linked glycosylation sites in HIV-1 89.6 surface antigen gp120 are indicated by ⋆ (complex-type) and x (high-mannose type). Positions of mutants N1-N7 and NV3 relative to variable loops V1-V5 are indicated on the top. Amino acid sequence of V2 (SEQ ID NO: 1) and V3 (SEQ ID NO: 2) loops and the specific sites changed in mutants N6, N7 and NV3 are indicated at the bottom.

FIG. 2A-2H: Neutralization sensitivity of WT HIV-1 89.6 and N-linked glycan mutants: Neutralization of pseudotyped virus generated with WT or mutant HIV-1 89.6 Env was performed as described in Example 1. Neutralization was quantified as % inhibition of viral infectivity measured by relative luciferase units (RLU) expressed in TZM-bl indicator cells. Data shown are representative results obtained from three individual experiments.

FIG. 3: CD4 independent infection by glycan mutants: Infectivity of HIV-1 pseudotyped with WT or mutant Env was measured in U87 cells expressing CXCR4. Inoculum used for each mutant virus was normalized by their infectivity in U87-CXCR4CD4 cells measured 200,000 relative luciferase units (RLU). Results represent the average and standard deviation obtained from three individual experiments.

FIG. 4: Immunization and challenge scheme.

FIG. 5: HIV-specific antibody responses after recombinant vaccinia and protein immunizations. HIV-1 SF162 gp120 (>95% pure, produced in stably transformed Chinese hamster ovary cells by Chiron Corporation and obtained from the NIH AIDS Research and Reference Reagent Program, Division of AIDS, NIAID, NIH) was used as the capturing antigen. Geometric mean endpoint titers of HIV-1 gp120-specific antibody response in immunized animals (N=6/group) were measured before immunization (“Preimmune”, Wk 0), two weeks after the second recombinant vaccinia virus immunization (“After vaccinia”, Wk 10), on the day the animals received the last immunization (“Before last boost”, Wk 77 for Groups 1 and 2 and Wk 66 for Groups 3 and Controls) and two weeks after the last immunization (“2 wk after last boost”, Wk 79 for Groups 1 and 2 and Wk 68 for Groups 3 and Controls). Vertical bars indicate standard deviations (Std Dev) within each group.

FIG. 6A-6B: Neutralizing antibody response on the day-of-challenge. (FIG. 6A) Neutralization titers were measured as the highest serum dilutions that resulted in 50% reduction of infectivity in the TZM-bl cell assay against replication competent viruses: homologous wild-type virus HIV-1 89.6, challenge virus SHIV89.6P-MN, or a heterologous virus HIV-1 SF162. The boxplots indicate the median as well as the range of responses, including the maximum, minimum, and the 75% and 25% quartiles as indicated. The p-values for the differences in response between Group 1 or Group 2 and the control group were ≦0.004 and ≦0.002, respectively (Mann-Whitney U test). The p-value for the differences between Groups 1 and 2 was ≦0.002 by the same test. (FIG. 6B) Neutralization of viruses pseudotyped with Env from twelve primary HIV-1 subtype B isolates by day-of-challenge serum from two animals immunized with the N7 mutant Env (Group 2). Neutralization was expressed as % reduction of RLU at 1:15 serum dilution with values from pre-immune sera subtracted. Data shown represent results obtained from two (FIG. 6B) or three (FIG. 6A) independent experiments.

FIG. 7A-7J: Plasma viral load (left panels) and peripheral blood CD4+ T-cells (right panels) in animals following SHIV89.6P-MN challenge. FIGS. 7A-7H show data for individual animals in each immunization or control group as indicated on the left margin. FIGS. 7I-7J show the mean and Std Dev of plasma viral load and peripheral blood CD4+ T-cells, respectively, in control and experimental groups. Symbol (X) denotes the approximate date of euthanasia due to AIDS.

FIGS. 8A-8D: Correlate of protection and survival curve of challenged animals. Log10 transformed 50% neutralization titer of each animal on the day of challenge against the challenge virus SHIV89.6P-MN was plotted against: (FIG. 8A) peak plasma viral load at wk 2, (FIG. 8B) setpoint viral load at wk 16, or (FIG. 8C) peripheral blood CD4+ T-cells at wk 28. (FIG. 8D) Kaplan-Meier analysis of AIDS-free survival after challenge infection. Correlation coefficients (Spearman's r value) and p-values (two-tailed) were calculated using Prism 4 (GraphPad) software.

FIG. 9: Graphs showing that removal of PNLG site at N197 increases sensitivity of a clade A primary isolate to neutralization by CD4IgG (left panel) or sCD4 (right panel). Percent neutralization is plotted as a function of concentration (μg/ml).

### DETAILED DESCRIPTION OF THE INVENTION

### Definitions

All scientific and technical terms used in this application have meanings commonly used in the art unless otherwise specified. As used in this application, the following words or phrases have the meanings specified.

As used herein, “polypeptide” includes proteins, fragments of proteins, and peptides, whether isolated from natural sources, produced by recombinant techniques or chemically synthesized. Polypeptides of the invention typically comprise at least about 6 amino acids, and can be at least about 15 amino acids. Typically, optimal immunological potency is obtained with lengths of 8-10 amino acids. Those skilled in the art also recognize that additional adjacent sequence from the original (native) protein can be included, and is often desired, in an immunologically effective polypeptide suitable for use as a vaccine. This adjacent sequence can be from 1, 2, 3, 4, 5, 6, 7, 8, 9 or 10 amino acids in length to as much as 15, 20, 25, 30, 35, 40, 45, 50, 75 or 100 amino acids in length or more.

As used herein, particularly in the context of polypeptides of the invention, “consisting essentially of” means the polypeptide consists of the recited amino acid sequence and, optionally, adjacent amino acid sequence. The adjacent sequence typically consists of additional, adjacent amino acid sequence found in the full length native protein, but variations from the native protein can be tolerated in this adjacent sequence while still providing an immunologically active polypeptide. Those skilled in the art recognize that certain variations are less likely than others to disrupt the structure, conformation and immunogenicity of the polypeptide.

As used herein, “epitope” refers to a molecular region of an antigen capable of eliciting an immune response and of being specifically recognized by the specific immune cell produced by such a response. Another term for “epitope” is “determinant” or “antigenic determinant”. Those skilled in the art often use the terms epitope and antigen interchangeably in the context of referring to the determinant against which an immune response is directed.

As used herein, “HIV” refers to human immunodeficiency virus. Unless otherwise indicated, references to sequences of HIV refer to the known wild type nucleic acid (or amino acid, as appropriate) sequence of HIV-1 isolate 89.6 (Collman, R., et al. 1992. J. Virol. 66:7517-7521; GenBank Accession No. U39362). The numbering of amino acid positions is based on Korber B et al., Numbering positions in HIV relative to HXB2CG. In: Human Retroviruses and AIDS (Korber B et al., eds.). Los Alamos National Laboratory, Los Alamos, N. Mex., 1998.

As used herein, “substitutional variant” refers to a molecule having one or more amino acid substitutions or deletions in the indicated amino acid sequence, yet retaining the ability to be “immunologically active”, or specifically recognized by an immune cell, or antibody. The amino acid sequence of a substitutional variant is typically at least 90% identical to the native amino acid sequence. Typically, the substitution is a conservative substitution.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric add, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “adjuvant” includes those adjuvants commonly used in the art to facilitate the stimulation of an immune response. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

As used herein, to “prevent” or “protect against” a condition or disease means to hinder, reduce or delay the onset or progression of the condition or disease.

### Overview

The present invention is based on the surprising discovery that immunization with an HIV envelope protein containing only a single glycan modification results in enhanced neutralizing antibody (NtAb) responses. Envelope modifications that result in enhanced neutralization sensitivity, while retaining the ability to mediate virus entry in a CD4 independent manner, represent a class of novel immunogens that can produce qualitatively and/or quantitatively different responses to HIV-1 than native proteins.

### Envelope Polypeptides

In one embodiment, the invention provides an isolated envelope polypeptide. The polypeptide comprises a hypervariable region (V1-V3) of gp120 having a mutation at amino acid residues 197-199 whereby a single N-linked glycan is removed. In a typical embodiment, the mutation replaces the asparagine (N) at residue 197 with another amino acid. The substituted amino acid can be glutamine (Q), creating an N197Q mutation. One example of a polynucleotide encoding a gp120 envelope protein having the N197Q mutation is described by SEQ ID NO: 5. Other amino acids, such as alanine (A) can be substituted for the asparagine as well, and still remove the N-linked glycan. Alternatively, one can modify the T or S at residue 199 to remove the glycosylation site. Because the N197 glycan is highly conserved among all HIV-1 subtypes, this approach is applicable across HIV-1 isolates. In a typical embodiment, the envelope protein comprises gp140 or gp160. Including at least the ectodomain of gp41 (gp41e) helps the gp120 trimer hold its form. Alternatively, one skilled in the art is aware of other means to enhance the conformation and stability of the gp120 protein.

A fragment of the invention consists of less than the complete amino acid sequence of the corresponding protein, but includes the recited epitope or antigenic region. As is understood in the art and confirmed by assays conducted using fragments of widely varying lengths, additional sequence beyond the recited epitope can be included without hindering the immunological response. In some cases, additional native sequence facilitates an optimal immune response. A fragment can be as few as 8 amino acids in length, or can encompass 10%, 20%, 30%, 40%, 50%, 60%, 70%, 80%, 90% or 95% of the full length of the protein.

The optimal length for the polypeptide of the invention will vary with the context and objective of the particular use, as is understood by those in the art. In some vaccine contexts, a full-length protein or large portion of the protein (e.g., up to 100 amino acids, 150 amino acids, 200 amino acids, 250 amino acids or more) provides optimal immunological stimulation, while in others, a short polypeptide (e.g., less than 50 amino acids, 40 amino acids, 30 amino acids, 20 amino acids, 15 amino acids or fewer) comprising the minimal epitope and/or a small region of adjacent sequence facilitates delivery and/or eases formation of a fusion protein or other means of combining the polypeptide with another molecule or adjuvant.

A polypeptide for use in a composition of the invention comprises a mutant gp120 polypeptide that contains an epitope or minimal stretch of amino acids sufficient to elicit an immune response. These polypeptides typically consist of such an epitope and, optionally, adjacent sequence. Those skilled in the art are aware that the epitope can still be immunologically effective with a small portion of adjacent wild type or other amino acid sequence present. Accordingly, a typical polypeptide of the invention will consist essentially of the recited mutant V1-V3 sequence and have a total length of up to 15, 20, 25 or 30 amino acids.

Env polyprotein precursor (gp160) N197Q (SEQ ID NO: 3) from Accession No. U39362 (strain 89.6, used in Examples below; modified here with N197Q mutation, highlighted):

```
    1 mrvkeirknw qhlrggilll gmlmicsaak ektwvtiyyg vpvwreattt lfcasdakay
  61 dtevhnvwat hacvptdpnp qevvlgnvte nfnmwknnmv dqmhediisl wdeslkpcvk
121 ltplcvtlnc tnlnitkntt nptssswgmm ekgeikncsf yittsirnkv kkeyalfnrl
181 dvvpientnn tkyrliscqt svitqacpkv sfqpipihyc vpagfamlkc nnktfngsgp
241 ctnvstvqct hgirpvvstq lllngslaee divizsenft dnaktiivql nesvvinctr
301 pnnntrrrls igpgrafyar rniigdirqa hcnisrakwn ntlqqivikl rekfrnktia
361 fnqssggdpe ivmhsfncgg effycntaql fnstwnvtgg tngtegndii tlqcrikqii
421 nmwqkvgkam yappitgqir cssnitglll trdggnstet eteifrpggg dmrdnwrsel
481 ykykvvriep igvaptrakr rtvqrekrav gigavflgfl gaagstmgaa svtltvqarl
541 llsgivqqqn nhlraieaqq hmlqltvwgi kqlqarvlal erylrdqqlm giwgcsgkli
601 cttsvpwnvs wsnksvddiw nnmtwmewer eidnytdyiy dlleksqtqq eknekellel
661 dkwaslwnwf ditnwlwyir lfimivggli glrivfavls ivnrvrqgys plsfqtllpa
721 srgpdrpegt eeeggerdrd rsgplvngfl alfwvdlrnl clflyhllrn lllivtrive
781 llgrrgweal kywwnliqyw sqelknsavs ilnatajava egtdrvikiv qracrairni
841 ptrirqgler all
```

Env polyprotein precursor (gp160) N197Q (SEQ ID NO: 4) from Accession No. AAB99976 (strain HXBc2, used for numbering convention; modified here with N197Q mutation, highlighted)

```
    1 mrvkekyqhl wrwgwrwgtm llgmlmicsa teklwvtvyy gvpvwkeatt tlfcasdaka
  61 ydtevhnvwa thacvptdpn pqevvlvnvt enfnmwkndm veqmhediis lwdqslkpcv
121 kltplcvslk ctdlkndtnt nsssgrmime kgeikncsfn istsirgkvq keyaffykld
181 iipidndtts ykltscqtsv itqacpkvsf epipihycap agfailkcnn ktfngtgpct
241 nvstvqcthg irpvvstqli ingslaeeev virsvnftdn aktiivqlnt sveinctrpn
301 nntrkririq rgpgrafvti gkignmrqah cnisrakwnn tikqiasklr eqfgnnktii
361 fkqssggdpe ivthsfncgg effycnstql fnstwfnstw stegsnnteg sdtitlpcri
421 kqiinmwqkv gkamyappis gqircssnit gllitrdggn snneseifrp gggdmrdnwr
481 selykykvvk ieplqvaptk akrrvvqrek ravgigalfl gflgaagstm gaasmtltvq
541 arqllsgivq qqnnllraie aqqhliqltv wgikqlqari iaverylkdq qllgiwgcsg
601 kiicttavpw naswsnksie qiwnhttwme wdreinnyts lihsiieesq nqqekneqel
661 leldkwaslw nwfnitnwlw yiklfimivg glvglrivfa vlsivnrvrq gysplsfqth
721 lptprgpdrp egieeegger drdrsirlvn gslaiiwddi rslclfsyhr lrdlilivtr
781 iveligrrgw eaikywwnll qywsqelkns avsllnatai avaegtdrvi evvqgacrai
841 rhiprrirqg lerill
```

Env gp120 N197Q (SEQ ID NO: 5) (from strain 89.6):

```
    1 mrvkeirknw qhlrggilll gmlmicsaak ektwvtiyyg vpvwreattt lfcasdakay
  61 dtevhnvwat hacvptdpnp qevvlgnvte nfnmwknnmv dqmhediisl wdeslkpcvk
121 ltplcvtlnc tnlnitkntt nptssswgmm ekqelkncsf ylttsirnkv kkeyalfnrl
181 dvvpientnn tkyrliscqt svitqacpkv sfqpipihyc vpagfamlkc nnktfngsgp
241 ctnvstvqct hgirpvvstq lllngslaee divirsenft dnaktiivql nesvvinctr
301 pnnntrrrls igpgrafyar rniigdirqa hcnisrakwn ntlqqivikl rekfrnktia
361 fnqssggdpe ivmhsfncgg effycntaql fnstwnvtgg tngtegndii tlqcrikqii
421 nmwqkvgkam yappitgqir cssnitglll trdggnstet eteifrpggg dmrdnwrsel
481 ykykvvriep iqvaptrakr rtvqrekr
```

In general, polypeptides (including fusion proteins) and polynucleotides as described herein are isolated. An “isolated” polypeptide or polynucleotide is one that is removed from its original environment. For example, a naturally occurring protein is isolated if it is separated from some or all of the coexisting materials in the natural system. An isolated polypeptide of the invention is one that has been isolated, produced or synthesized such that it is separate from a complete, native protein in the virus of origin, although the isolated polypeptide may subsequently be expressed by a recombinant virus or other vector. A recombinant virus that comprises a polynucleotide of the invention is an example of subject matter provided by the invention. Preferably, such isolated polypeptides are at least about 90% pure, more preferably at least about 95% pure and most preferably at least about 99% pure. A polynucleotide is considered to be isolated if, for example, it is cloned into a vector that is not part of the natural environment.

The polypeptide can be isolated from its naturally occurring form, produced by recombinant means or synthesized chemically. Recombinant polypeptides encoded by DNA sequences described herein can be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, insect, and higher eukaryotic cells. Preferably the host cells employed are E. coli, yeast or a mammalian cell line such as Cos or CHO. Supernatants from the soluble host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Fragments and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, wherein amino acids are sequentially added to a growing amino acid chain (Merrifield, 1963, J. Am. Chem. Soc. 85:2146-2149). Equipment for automated synthesis of polypeptides is commercially available from suppliers such as Perkin Elmer/Applied BioSystems Division (Foster City, Calif.), and may be operated according to the manufacturer's instructions.

Variants of the polypeptide for use in accordance with the invention can have one or more amino acid substitutions, deletions, additions and/or insertions in the amino acid sequence indicated that result in a polypeptide that retains the ability to elicit an immune response to HIV or HIV-infected cells. Such variants may generally be identified by modifying one of the polypeptide sequences described herein and evaluating the reactivity of the modified polypeptide using a known assay such as a T cell assay described herein. Polypeptide variants preferably exhibit at least about 90%, and most preferably at least about 95% identity to the identified polypeptides. These amino acid substitutions include, but are not necessarily limited to, amino acid substitutions known in the art as “conservative”.

A “conservative” substitution is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

One can readily confirm the suitability of a particular variant by assaying the ability of the variant polypeptide to elicit an immune response. The ability of the variant to elicit an immune response can be compared to the response elicited by the parent polypeptide assayed under identical circumstances. One example of an immune response is a cellular immune response. The assaying can comprise performing an assay that measures T cell stimulation or activation. Examples of T cells include CD4 and CD8 T cells.

One example of a T cell stimulation assay is a cytotoxicity assay, such as that described in Koelle, D M et al., Human Immunol. 1997, 53; 195-205. In one example, the cytotoxicity assay comprises contacting a cell that presents the antigenic viral peptide in the context of the appropriate HLA molecule with a T cell, and detecting the ability of the T cell to kill the antigen presenting cell. Cell killing can be detected by measuring the release of radioactive ⁵¹Cr from the antigen presenting cell. Release of ⁵¹Cr into the medium from the antigen presenting cell is indicative of cell killing. An exemplary criterion for increased killing is a statistically significant increase in counts per minute (cpm) based on counting of ⁵¹Cr radiation in media collected from antigen presenting cells admixed with T cells as compared to control media collected from antigen presenting cells admixed with media.

### Fusion Proteins

The polypeptide can be a fusion protein. In one embodiment, the fusion protein is soluble. A soluble fusion protein of the invention can be suitable for injection into a subject and for eliciting an immune response. Within certain embodiments, a polypeptide can be a fusion protein that comprises multiple polypeptides as described herein, or that comprises at least one polypeptide as described herein and an unrelated sequence. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985, Gene 40:39-46; Murphy et al., 1986, Proc. Natl. Acad. Sci. USA 83:8258-8262; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptides. Similarly, stop codons required to end translation and transcription termination signals are present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al., 1997, New Engl. J. Med., 336:86-9).

In some embodiments, it may be desirable to couple a therapeutic agent and a polypeptide of the invention, or to couple more than one polypeptide of the invention. For example, more than one agent or polypeptide may be coupled directly to a first polypeptide of the invention, or linkers that provide multiple sites for attachment can be used. Alternatively, a carrier can be used. Some molecules are particularly suitable for intercellular trafficking and protein delivery, including, but not limited to, VP22 (Elliott and O'Hare, 1997, Cell 88:223-233; see also Kim et al., 1997, J. Immunol. 159:1666-1668; Rojas et al., 1998, Nature Biotechnology 16:370; Kato et al., 1998, FEBS Lett. 427(2):203-208; Vives et al., 1997, J. Biol. Chem. 272(25):16010-7; Nagahara et al., 1998, Nature Med. 4(12):1449-1452).

A carrier may bear the agents or polypeptides in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088).

### Polynucleotides, Vectors, Host Cells and Recombinant Viruses

The invention provides polynucleotides that encode one or more envelope polypeptides of the invention. The complete genome sequence of HIV-1 strain 89.6, has been deposited with Genbank, Accession No. U39362. The coding sequence for the envelope protein is at bases 6223-8784 (SEQ ID NO: 6), with gp120 encoded by bases 6223-7746 (SEQ ID NO: 7) and gp41 encoded by bases 7747-8781 (SEQ ID NO: 8).

The polynucleotide of the invention can be included in a vector. The vector can further comprise an expression control sequence operably linked to the polynucleotide of the invention. In some embodiments, the vector includes one or more polynucleotides encoding other molecules of interest. In one embodiment, the polynucleotide of the invention and an additional polynucleotide can be linked so as to encode a fusion protein.

HIV-1 89.6 coding sequence for env (SEQ ID NO: 6):

6223 caatgagagt gaaggagatc
6241 aggaagaatt ggcagcactt gagagggggc atcttgctcc ttgggatgtt gatgatctgt
6301 agtgctgcaa aagaaaaaac gtgggtcaca atctattatg gggtacctgt gtggagagaa
6361 gcaaccacca ctctattttg tgcatcagat gctaaagcct atgatacaga ggtacataat
6421 gtttgggcca cacatgcctg tgtacccaca gaccccaacc cacaagaagt agtattggga
6481 aatgtgacag aaaattttaa catgtggaaa aataacatgg tagatcagat gcatgaggat
6541 ataatcagtt tatgggatga aagcctaaag ccatgtgtaa aattaacccc actctgtgtt
6601 actttaaatt gcactaattt gaatatcact aagaatacta ctaatcccac tagtagcagc
6661 tggggaatga tggagaaagg agaaataaaa aattgctctt tctatatcac cacaagcata
6721 agaaataagg taaagaaaga atatgcactt tttaatagac ttgatgtagt accaatagaa
6781 aatactaata atactaagta taggttaata agttgtaaca cctcagtcat tacacaggcc
6841 tgtccaaagg tatcctttca gccaattccc atacattatt gtgtcccggc tgggtttgcg
6901 atgctaaagt gtaacaataa gacattcaat ggatcaggac catgcacaaa tgtcagcaca
6961 gtacaatgta cacatggaat taggccagtg gtgtcaactc aactgctgtt aaatggcagt
7021 ctagcagaag aagacatagt aattagatct gaaaatttca cagacaatgc taaaaccata
7081 atagtacagc taaatgaatc tgtagtaatt aattgtacaa gacccaacaa caatacaaga
7141 agaaggttat ctataggacc agggagagca ttttatgcaa gaagaaacat aataggagat
7201 ataagacaag cacattgtaa cattagtaga gcaaaatgga ataacacttt acaacagata
7261 gttataaaat taagagaaaa atttaggaat aaaacaatag cctttaatca atcctcagga
7321 ggggacccag aaattgtaat gcacagtttt aattgtggag gggaattttt ctactgtaat
7381 acagcacaac tgtttaatag tacttggaat gttactggag ggacaaatgg cactgaagga
7441 aatgacataa tcacactcca atgcagaata aaacaaatta taaatatgtg gcagaaagta
7501 ggaaaagcaa tgtatgcccc tcccatcaca ggacaaatta gatgttcatc aaatattaca
7561 gggctgctac taacaagaga tggaggtaat agtactgaga ctgagactga gatcttcaga
7621 cctggaggag gagatatgag ggacaattgg agaagtgaat tatataaata taaagtagta
7681 agaattgaac caataggagt agcacccacc agggcaaaga gaagaacagt gcaaagagaa
7741 aaaagagcag tgggaatagg agctgtgttc cttgggttct tgggagcagc aggaagcact
7801 atgggcgcag cgtcagtgac gctgacggta caggccaggc tattattgtc tggtatagtg
7861 cagcagcaga acaatctgct gagggctatt gaggcgcaac agcatatgtt gcaactcaca
7921 gtctggggca tcaagcagct ccaggcaaga gtcctggctc tggaaagata cctaagggat
7981 caacagctca tgggaatttg gggttgctct ggaaaactca tttgcaccac ttctgtgcct
8041 tggaatgtta gttggagtaa taaatctgtg gatgatattt ggaataacat gacctggatg
8101 gagtgggaaa gagaaattga caattacaca gactatatat atgacttact tgaaaaatcg
8161 caaacccaac aagaaaagaa tgaaaaagaa ttattggaat tggataaatg ggcaagtttg
8221 tggaattggt ttgacataac aaactggctg tggtatataa gattattcat aatgatagta
8281 ggaggcttga taggtttaag aatagttttt gctgtacttt ctatagtaaa tagagttagg
8341 cagggatatt caccattatc gtttcagacc ctcctcccag cctcgagggg acccgacagg
8401 cccgaaggaa cagaagaaga aggtggagag agagacagag acagatccgg tccattagtg
8461 aacggattct tggcactttt ctgggtcgat ttgaggaacc tgtgcctctt cctctaccac
8521 ctcttgagaa acttactctt gattgtaacg aggattgtgg aacttctggg acgcaggggg
8581 tgggaagccc tcaaatattg gtggaatctc ctgcaatatt ggagccagga actaaagaat
8641 agtgctgtta gcttgctcaa cgccacagcc atagcagtag ctgaggggac agatagggtt
8701 ataaaaatag tacaaagagc ttgtagagct attcgcaaca tacctacaag aatcagacag
8761 ggcttggaaa gggctttgct ataa

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, alphavirus, vaccinia or a pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

The invention also provides a host cell transformed with a vector of the invention. The transformed host cell can be used in a method of producing a polypeptide of the invention. The method comprises culturing the host cell and recovering the polypeptide so produced. The recovered polypeptide can be purified from culture supernatant.

Vectors of the invention can be used to genetically modify a cell, either in vivo, ex vivo or in vitro. Several ways of genetically modifying cells are known, including transduction or infection with a viral vector either directly or via a retroviral producer cell, calcium phosphate precipitation, fusion of the recipient cells with bacterial protoplasts containing the DNA, treatment of the recipient cells with liposomes or microspheres containing the DNA, DEAE dextran, receptor-mediated endocytosis, electroporation, micro-injection, and many other techniques known to those of skill. See, e.g., Sambrook et al. Molecular Cloning—A Laboratory Manual (2nd ed.) 1-3, 1989; and Current Protocols in Molecular Biology, F. M. Ausubel et al., eds., Greene Publishing Associates, Inc. and John Wiley & Sons, Inc., (1994 Supplement).

Examples of viral vectors include, but are not limited to retroviral vectors based on, e.g., HIV, SIV, and murine retroviruses, gibbon ape leukemia virus and other viruses such as adeno-associated viruses (AAVs) and adenoviruses. (Miller et al. 1990, Mol. Cell Biol. 10:4239; J. Kolberg 1992, NIH Res. 4:43, and Cometta et al. 1991, Hum. Gene Ther. 2:215). Widely used retroviral vectors include those based upon murine leukemia virus (MuLV), gibbon ape leukemia virus (GaLV), ecotropic retroviruses, simian immunodeficiency virus (SIV), human immunodeficiency virus (HIV), and combinations. See, e.g. Buchscher et al. 1992, J. Virol. 66(5):2731-2739; Johann et al. 1992, J. Virol. 66(5):1635-1640; Sommerfelt et al. 1990, Virol. 176:58-59; Wilson et al. 1989, J. Virol. 63:2374-2378; Miller et al. 1991, J. Virol. 65:2220-2224, and Rosenberg and Fauci 1993 in Fundamental Immunology, Third Edition, W. E. Paul (ed.) Raven Press, Ltd., New York and the references therein; Miller et al. 1990, Mol. Cell. Biol. 10:4239; R. Kolberg 1992, J. NIH Res. 4:43; and Cometta et al. 1991, Hum. Gene Ther. 2:215.

In vitro amplification techniques suitable for amplifying sequences to be subcloned into an expression vector are known. Examples of such in vitro amplification methods, including the polymerase chain reaction (PCR), ligase chain reaction (LCR), Qβ-replicase amplification and other RNA polymerase mediated techniques (e.g., NASBA), are found in Sambrook et al. 1989, Molecular Cloning—A Laboratory Manual (2nd Ed) 1-3; and U.S. Pat. No. 4,683,202; PCR Protocols A Guide to Methods and Applications (Innis et al. eds.) Academic Press Inc. San Diego, Calif. 1990. Improved methods of cloning in vitro amplified nucleic acids are described in U.S. Pat. No. 5,426,039.

The invention additionally provides a recombinant microorganism genetically modified to express a polynucleotide of the invention. The recombinant microorganism can be useful as a vaccine, and can be prepared using techniques known in the art for the preparation of live attenuated vaccines. Examples of microorganisms for use as live vaccines include, but are not limited to, viruses and bacteria. In a preferred embodiment, the recombinant microorganism is a virus. Examples of suitable viruses include, but are not limited to, vaccinia virus and other poxviruses, adenovirus, adeno-associated virus, retrovirus and alphavirus.

### Compositions

The invention provides compositions that are useful for treating and preventing HIV infection. The compositions can be used to inhibit viral replication and to kill virally-infected cells. In one embodiment, the composition is a pharmaceutical composition. The composition can comprise a therapeutically or prophylactically effective amount of a polypeptide, polynucleotide, recombinant virus, APC or immune cell of the invention. An effective amount is an amount sufficient to elicit or augment an immune response, e.g., by activating T cells or eliciting production of neutralizing antibodies. One measure of the activation of T cells is a cytotoxicity assay, as described in D. M. Koelle et al., 1997, Human Immunol. 53:195-205. In some embodiments, the composition is a vaccine.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other viral proteins may be present, either incorporated into a fusion polypeptide or as a separate compound, within the composition or vaccine.

A pharmaceutical composition or vaccine may contain DNA encoding one or more of the polypeptides of the invention, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, 1998, Crit. Rev. Therap. Drug Carrier Systems 15:143-198, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., 1989, Proc. Natl. Acad. Sci. USA 86:317-321; Flexner et al., 1989, Ann. My Acad. Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91102805; Berkner, 1988, Biotechniques 6:616-627; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art. The DNA may also be “naked,” as described, for example, in Ulmer et al., 1993, Science 259:1745-1749 and reviewed by Cohen, 1993, Science 259:1691-1692. The uptake of naked DNA may be increased by coating the DNA onto biodegradable beads, which are efficiently transported into the cells.

Certain embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic immunity (see Timmerman and Levy, Ann. Rev. Med. 50:507-529, 1999). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural killer cells (CD56), as determined using standard assays. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (Zitvogel et al., 1998, Nature Med. 4:594-600).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritc cells.

Dendritic cells are conveniently categorized as “immature” and “mature” cells, which allows a simple way to discriminate between two well-characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costmulatory molecules (e.g., CD40, CD80 and CD86).

APCs may generally be transfected with a polynucleotide encoding a polypeptide (or portion or other variant thereof) such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as humans and primates, as well as other veterinary subjects. Preferably, the patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The compositions are administered in any suitable manner, often with pharmaceutically acceptable carriers. Suitable methods of administering cells in the context of the present invention to a patient are available, and, although more than one route can be used to administer a particular cell composition, a particular route can often provide a more immediate and more effective reaction than another route.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases such as HIV infection, the physician needs to evaluate the production of an immune response against the virus, progression of the disease, and any treatment-related toxicity.

For example, a vaccine or other composition containing a subunit envelope protein can include 1-10,000 micrograms of envelope protein per dose. In a preferred embodiment, 10-1000 micrograms of envelope protein is included in each dose in a more preferred embodiment 10-100 micrograms of envelope protein per dose. Preferably, a dosage is selected such that a single dose will suffice or, alternatively, several doses are administered over the course of several months. For compositions containing HIV polynucleotides or peptides, similar quantities are administered per dose.

In one embodiment, between 1 and 10 doses may be administered over a 52 week period. Preferably, 6 doses are administered, at intervals of at least 3 weeks, or up to every 3-4 months, and booster vaccinations may be given periodically thereafter. Alternate protocols may be appropriate for individual patents. A suitable dose is an amount of a compound that, when administered as described above, is capable of promoting an antiviral immune response, and is at least 10-50% above the basal (i.e., untreated) level. Such vaccines should also be capable of causing an immune response that leads to an improved clinical outcome in vaccinated patients as compared to non-vaccinated patients. In general, for pharmaceutical compositions and vaccines comprising one or more polypeptides, the amount of each polypeptide present in a dose ranges from about 0.1 μg to about 5 mg per dose. Preferably, the amount ranges from about 10 to about 1000 μg per dose. Suitable volumes for administration will vary with the size, age and immune status of the patient, but will typically range from about 0.1 mL to about 5 mL, with volumes less than about 1 mL being most common.

Compositions comprising immune cells are preferably prepared from immune cells obtained from the subject to whom the composition will be administered. Alternatively, the immune cells can be prepared from an HLA-compatible donor. The immune cells are obtained from the subject or donor using conventional techniques known in the art, exposed to APCs modified to present an epitope of the invention, expanded ex vivo, and administered to the subject. Protocols for ex vivo therapy are described in Rosenberg et al., 1990, New England J. Med. 9:570-578. In addition, compositions can comprise APCs modified to present an epitope of the invention.

Immune cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to enrich and rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., 1997, Immunological Reviews 157:177).

Administration by many of the routes of administration described herein or otherwise known in the art may be accomplished simply by direct administration using a needle, catheter or related device, at a single time point or at multiple time points.

### Methods of Treatment and Prevention

The invention provides a method for treatment and/or prevention of HIV infection in a subject. The method comprises administering to the subject a composition of the invention. The composition can be used as a therapeutic or prophylactic vaccine. In one embodiment, the HIV is HIV-1. The invention additionally provides a method for inhibiting viral replication, for reducing plasma levels of HIV in a subject, for maintaining CD4 cells in a subject, for prolonging disease-free survival in an HIV-infected subject, for killing virally-infected cells, for increasing secretion of lymphokines having antiviral and/or immunomodulatory activity, and for enhancing production of virus-specific and virus-neutralizing antibodies. The method comprises contacting an infected cell with an immune cell directed against an antigen of the invention, for example, as described in the Examples presented herein. The contacting can be performed in vitro or in vivo. In one embodiment, the immune cell is a T cell. T cells include CD4 and CD8 T cells. In another embodiment, the immune cell is a B cell. Compositions of the invention can also be used as a tolerizing agent against immunopathologic disease.

In addition, the invention provides a method of producing immune cells directed against HIV. The method comprises contacting an immune cell with a polypeptide of the invention. The immune cell can be contacted with the polypeptide via an antigen-presenting cell, wherein the antigen-presenting cell is modified to present an antigen included in a polypeptide of the invention. Preferably, the antigen-presenting cell is a dendritic cell. The cell can be modified by, for example, peptide loading or genetic modification with a nucleic acid sequence encoding the polypeptide. In one embodiment, the immune cell is a T cell. T cells include CD4 and CD8 T cells. Also provided are immune cells produced by the method. The immune cells can be used to inhibit viral replication, to kill virally-infected cells, in vitro or in vivo, to increase secretion of lymphokines having antiviral and/or immunomodulatory activity, to enhance production of virus-specific antibodies, or in the treatment or prevention of viral infection in a subject.

### EXAMPLES

The following examples are presented to illustrate the present invention and to assist one of ordinary skill in making and using the same. The examples are not intended in any way to otherwise limit the scope of the invention.

### Example 1

### Removal of a Single N-Linked Glycan in HIV-1 gp120 Results in Enhanced Ability to Induce Neutralizing Antibody Responses

This example examines the role of specific glycans. Single or multiple mutations were introduced into potential N-linked glycosylation sites in hypervariable regions (V1-V3) of the env gene of HIV-1 89.6. Three mutants tested showed enhanced sensitivity to soluble CD4. Mutant N7 (N197Q) in the carboxy-terminal stem of the V2 loop showed the most pronounced increase in sensitivity to broadly neutralizing antibodies (NtAb), including those targeting the CD4-binding site (IgG1b12) and the V3 loop (447-52D). This mutant is also sensitive to CD4-induced NtAb17b in the absence of CD4. Unlike wild-type (WT) Env, mutant N7 mediates CD4-independent infection in U87-CXCR4 cells. To study the immunogenicity of mutant Env, we immunized pig-tailed macaques with recombinant vaccinia viruses, one expressing SIVmac239 Gag-Pol, and the other, HIV-1 89.6 Env gp160 in WT or mutant forms. Animals were boosted 1416 months later with SIV gag DNA and the cognate gp140 protein before intrarectal challenge with SHIV89.6P-MN. Day-of-challenge sera from animals immunized with mutant N7 Env had significantly higher and broader neutralizing activities than sera from WT Env-immunized animals. Neutralizing activity was observed against SHIV89.6, SHIV89.6P-MN, HIV-1 SF162, and a panel of subtype B primary isolates. Compared to control animals, immunized animals showed significant reduction of plasma viral load and increased survival after challenge, which correlated with prechallenge NtAb titers. These results indicate the potential advantages for glycan modification in vaccine design.

Various references are cited throughout this example by numerals in parentheses. The corresponding citations can be found in a numbered list at the end of this example.

### Materials and Methods

Cells: The U87 human astroglioma cell line stably transduced with human CXCR4 (14) was cultured in Dulbecco's modified Eagle medium (DMEM) supplemented with 10% FBS, penicillin (100 U/ml), streptomycin (100 μg/ml), glutamine (2 mM) and selected with 1 ug/ml puromycin. The U87-CD4/CXCR4 cells obtained through the AIDS Research and Reference Reagent Program (ARRRP, Catalog No. 4036) from HongKui Deng and Dan R. Littman were cultured in the same medium except containing 15% of FBS and selected with puromycin (1 μg/ml) plus G418 (300 μg/ml). JC53-LB cells (referred to as TZM-bl cells herein) contributed by John Kappes and Xiaoyun Wu were also obtained from NIH AIDS Research and Reference Reagent Program (ARRRP, Catalog No. 8129). Both TZM-bl and 293T (ATCC Catalog No. 11268) cells were cultured in DMEM supplemented with 10% of FBS, penicillin (100 U/ml), streptomycin (100 μg/ml), and glutamine (2 mM). The CEMx174 cells (46) were maintained as a suspension culture in RPMI 1640 medium with 10% of FBS plus penicillin, streptomycin and glutamine.

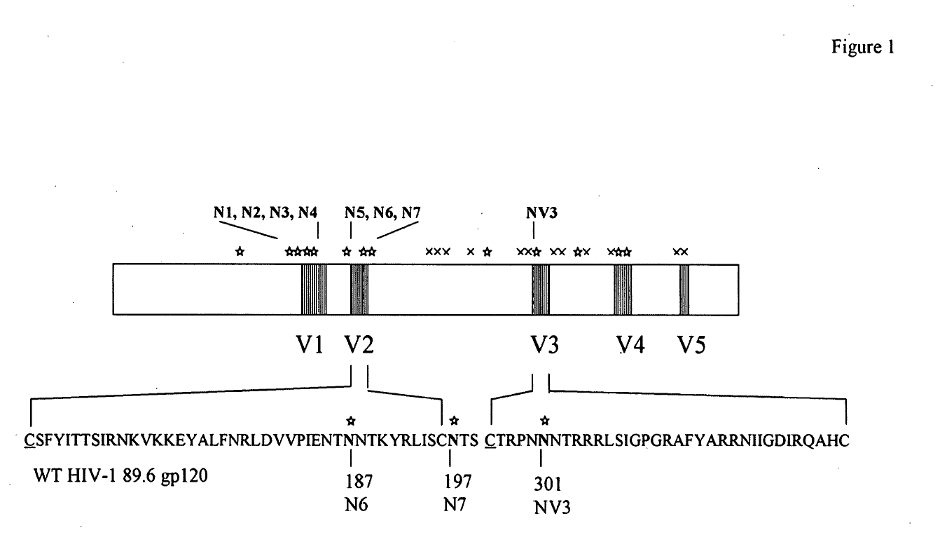

Construction of N-linked glycosylation site mutants: Plasmid pSL1180 containing the rev-env gene of HIV-1 89.6 was used as the template for site-directed mutagenesis with the QuickChange mutagenesis kit (Stratagene). A conservative substitution from asparagine (N) to glutamine (Q) was introduced in potential N-linked glycosylation sites (N—X—S/T) in variable regions V1/V2 and V3 of the env gene (FIG. 1). Primers used for the mutagenesis were: (1). N6 mutant: 5′-GTAGTACCAATAGAAAATACTCAGAATACTAAGTATAGG-3′ (forward; SEQ ID NO: 9) and 5′-CCTATACTTAGTATTCTGAGTATTTTCTATTGGTACTAC-3′ (reversed; SEQ ID NO: 10); (2). N7 mutant: 5′-GGTTAATAAGTTGTCAGACCTCAGTCATTACACAGGCC-3′ (forward; SEQ ID NO: 11) and 5′-GGCCTGTGTAATGACTGAGGTCTGACAACTTATTAACC-3′ (reversed; SEQ ID NO: 12); (3). NV3 mutant: 5′-GTACAAGACCCAACCAGAATACAAGAAGAAGGTTATC-3′ (forward; SEQ ID NO: 13), 5′-GATAACCTTCTTCTTGTATTCTGGTTGGGTCTTGTAC-3′ (reversed; SEQ ID NO: 14). The N67V3 mutation was made by inserting the Bgl II fragment (nt. 7164 to nt. 7729) containing the NV3 mutation into the N67 containing plasmid. The mutated env genes were then subcloned into mammalian expression vector pCl-neo. The PCR cycling parameters for the mutagenesis included two steps: step 1, 95° C., 30 sec; step 2, 18 cycles of 95° C., 30 sec followed by 52° C., 1 min and then 68° C., 18 min. Results of site-directed mutagenesis were verified by DNA sequencing.

Monoclonal antibodies: The following monoclonal antibodies were obtained from NIH ARRRP: 4E10, 2F5 (5, 38, 39), and 2G12 (5, 52) from Kermann Katinger, IgG1b12 from Dennis Burton and Carlos Barbas (2, 7, 8, 44), 447-52D (12, 19-21, 35, 58) from Susan Zolla-Pazner, and 17b (28, 48, 50, 51; 53, 54), from James E. Robinson. Recombinant soluble CD4-183, also obtained from the same source, contains the first 2 domains of human CD4 produced in E. coli.

Determination of co-receptor usage by complementation analysis: Complementation assay with pseudotyped HIV-1 was performed as previously described (30, 32). Briefly, pseudotyped viruses carrying WT or mutant 89.6 envelope glycoproteins were produced by 293T cells after co-transfection with pCl-neo (rev-env) expressing the env gene of interest and an env-deficient HIV-1 backbone vector pNL4.3LucR⁻E⁻ expressing the firefly luciferase gene. Two days after transfection, culture supernatants containing pseudotyped virus were collected and filtered through a 0.45 micron filter before use for infectivity assays. U87-CXCR4 or U87-CD4/CXCR4 cells were seeded at 1×10⁴ cells per well in a 96-well plate overnight and treated with polybrene (2 μg/ml) for 30 min prior to infection by pseudotyped viruses. Two hours after inoculation, inoculum was replaced with 200 μl of fresh medium without any antibiotics and infection continued for a total of 48 hours. Infectivity was measured as relative luminescence units (RLU) in infected cell cultures quantified by the SteadyLite HTS Luminescence Reporter Gene Assay System (PerkinElmer). To compare the relative infectivity of WT and mutant 89.6 in U87-CXCR4 cells, we used inoculum containing the same infectivity (RLU=2.0×10⁵) as determined in U87-CD4/CXCR4 cells.

Neutralization assay. Neutralization was measured as a function of reductions in luciferase reporter gene expression after infection of TZM-bl indicator cells, either with replication competent virus, or with Env-pseudotyped virus, as previously described (29, 33). Briefly, 200 TCID₅₀ of virus was incubated either with a single dilution (1:15), or with serial 3-fold dilutions of serum samples in triplicate in a total volume of 150 μL for 1 hr at 37° C. in 96 well flat-bottom culture plates. Freshly trypsinized cells (10,000 cells in 100 μl of growth medium containing 75 μg/mL DEAE dextran) were added to each well. One set of control wells received cells+virus (virus control) and another set received cells only (background control). After a 48 hour incubation, 100 μL of cells were transferred to a 96-well black solid plates (Costar) for assays of luciferase activities using Bright Glo substrate solution as described by the supplier (Promega). Assay stocks of Env-pseudotyped viruses were prepared by transfection in 293T cells (29), whereas stocks of replication competent viruses SHIV 89.6, SHIV 89.6P-MN and HIV-1 SF162 were prepared in human PBMC. All assay stocks were titrated in TZM-bl cells as described (29). Neutralization activity was expressed either as % reduction of RLU for experiments with single (1:15) serum dilutions, or as the serum dilution that resulted in 50% reduction of RLU in serial dilution experiments. Values obtained with pre-immune sera were subtracted from those obtained with immune sera for each animal.

Immunogens: Four recombinant vaccinia viruses were used in this study: 1. recombinant v-ELgp160(89.6) expresses the full length WT env gene of HIV-1 89.6 (11); 2. v-ELgp160(89.6)N7 expresses the same env gene with a single glycan mutation N7; 3. v-ELgp160(89.6)N67V3 expresses the env gene with triple glycan mutations N6, N7 and NV3; 4. recombinant v-ELgag/pol(SIVmac239) expresses the native gag-pol genes of SIVmac239. Expression of the transgene was under the control of a synthetic early-late promoter of vaccinia virus (9). The transgene was inserted into the thymidine kinase gene of the WR strain of vaccinia virus. Construction and propagation of these viruses was as previously described (22-24). Expression of the transgene was verified by Western blot analysis. Plasmid DNA expressing codon-optimized SIVmac239 gag gene was kindly provided by Dr. J. Shiver (7, 38) and was prepared with EndoFree Maxiprep kit (Qiagen). Protein immunogens used in this study were produced in African green monkey kidney cells (BSC-40) infected with recombinant vaccinia virus expressing SIVmac239 Gag-Pol particles, or HIV-1 89.6 Env gp140, in WT, N7, or N67V3 forms. SIVmac239 Gag-Pol particles were purified from infected cell supernatant by ultracentrifugation as described (23, 36). Recombinant vaccinia viruses expressing HIV-1 89.6 Env gp140 were constructed as described above, with the exception that two stop codons were introduced to substitute amino acids 680 and 681 (isoleucine and arginine) immediately upstream of the hydrophobic anchor sequence in gp41. WT or mutant gp140 was purified from infected BSC-40 cell supernatant by lentil lectin affinity and size exclusion chromatography as described (25).

Immunization: Twenty-four juvenile pig-tailed macaques (M. nemestrina), all tested negative for simian type D retrovirus by serology and PCR, were used for this study. Macaques in the experimental arm (total of 18, in three groups of 6 each) were inoculated by skin scarification at wk 0 and wk 8 with 10⁸ plaque-forming-units (PFU) of two recombinant viruses, one expressing SIVmac239 Gag-Pol and the other expressing one of three forms of HIV-1 89.6 Env: WT, N7 or N67V3. All animals were boosted with DNA plasmid expressing SIV Gag (17) and the cognate gp140 Env protein (WT, N7 or N67V3). DNA (5.0 mg/dose in PBS) was administered by intramuscular injection at four sites. Env gp140 (100 ug/dose) was adjuvanted with 0.025% Alhydrogel and was also administered intramuscularly. Control animals (N=6) were primed with parental vaccinia virus WR and boosted with vector plasmid and adjuvant only. To accommodate the large size of the study, we performed the booster immunization (and subsequent challenge) in two stages, separated by 2 months. Animals in Groups 1 and 2 were rested for 16 months between the second vaccinia virus inoculation and the final immunization, whereas those in Group 3 and the control group were rested for 14 months.

SHIV challenge: Four weeks after the booster immunization, all macaques were challenged intrarectally with SHIV89.6P-MN. The challenge virus was derived from a macaque-passaged SHIV89.6P stock (43) (kindly provided by Dr. N. Letvin), after two passages in CD8⁺-depleted peripheral blood mononuclear cells (PBMC) from M. nemestrina (15). Intrarectal inoculation was performed atraumatically twice, 1 h apart, with 1 mL of the undiluted challenge virus containing 25 50% animal infectious dose (AID₅₀). Animals were monitored for body weight, temperature and general health and tissue samples collected periodically for in vitro analyses. Euthanasia was performed according to the following criteria: AIDS; termination of experiment; or deteriorating physical condition for reasons unrelated to infection. Euthanasia was considered to be AIDS related if the animal exhibited peripheral blood CD4⁺ T cell depletion (<200/mm³) and two or more of the following conditions: wasting, untreatable diarrhea, opportunistic infections, proliferative diseases (e.g. lymphoma), and abnormal hematology (e.g., anemia, thrombocytopenia or leukopenia). All procedures involving live animals or tissues are performed with the approval of the Institutional Animal Care and Use Committee.

Viral load determination: Plasma viral load was determined by real-time reverse transcription (RT)-polymerase chain reaction (PCR) based on methods originally described by (49). Briefly, viral RNA was prepared from EDTA-anticoagulated, cell-free plasma using the Gentra Puregene RNA isolation kit according to the manufacturers instructions (Gentra Systems). RNA was precipitated in the presence of glycogen, resuspended in 50 μL of nuclease-free water and analyzed immediately. Oligonucleotides were chosen within the SIV gag sequence. The primers GAG5f (5′-ACTTTCGGTCTTAGCTCCATTAGTG-3′; SEQ ID NO: 15), GAG3r (5′-TTTTGCTTCCTCAGTGTGTTTCA-3′; SEQ ID NO: 16), and the TaqMan probe GAG1tq (5′-TTCTCTTCTGCGTGAATGCACCAGATGA-3′; SEQ ID NO: 17) were all obtained from Applied Biosystems. In the probe the fluorescence reporter dye at the 5′ end was FAM (6-carboxyfluorescein) and the quencher dye at the 3′ end was TAMRA (6-carboxytetramethyl-rhodamine). A two-step RT-PCR using the TaqMan Gold RT-PCR Kit (Applied Biosystems) was performed. The control template is an in vitro transcript containing KpnI-BamHI SIV gag fragment from SIVmac239, prepared from the plasmid pSIV-BS6 kindly provided by Dr. J. Lifson. RNA transcripts were diluted in nuclease-free water and stored at −80° C. in single use aliquots. For each run, an RNA standard curve was generated from triplicate samples of purified pSIV-BS-derived in vitro transcript, ranging from 3×10⁶ to nominal copy equivalents/reaction. For each test sample, four reactions were run. Triplicate aliquots were reverse transcribed and amplified plus one aliquot that was processed without addition of reverse transcriptase. The performance of the assay showed at least 5 log₁₀ linear dynamic range, with typical R² values for plots of threshold cycles vs. log₁₀ input copy number of >0.98.

Immunophenotype and hematologic analyses: Absolute numbers of circulating CD3/4⁺ cells are determined via a two part process according to Centers for Disease Control and Prevention (CDC) regulations for determining circulating CD3/4⁺ cells. Briefly, 1 mL whole blood was treated with 14 mL ammonium chloride lysis solution for 7 minutes, after which time the mixture was centrifuged for 5 minutes at 700 g and supernatant discarded. The resultant cell pellet was then resuspended in 1 ml staining medium (RPMI supplemented with 1% FBS and 0.02% NaN3) and 50 μl aliquots of the cell suspension were then tri-stained with FITC labeled anti-CD3 (clone SP34-2), PerCp-Cy5.5-labeled anti-CD4 (clone L200) and APC-labeled anti-CD8 (clone SK1), and doubled stained with FITC-labeled anti-CD2 (clone S5.2) and PerCP-Cy5.5-labeled anti-CD20 (clone L27); unstained cells were used as staining controls. All antibodies were purchased from Becton Dickinson Immunocytometry Systems (BDIS). Cells were then incubated in the dark for 20-30 minutes after which time they were washed with 200 μL of PBS, and then transferred to FACS tubes (Falcon) in a final volume of 230 μl of 1% paraformaldehyde and run on a four-color flow cytometer (FACSCalibur, BDIS) where a minimum of 10,000 gated lymphocyte events were collected. The absolute numbers of CD3⁺/4⁺ and other relevant T and B cell populations were determined via off-line analysis using flow cytometry analysis software following the CDC guidelines for T cell determinations in HIV-infected individuals.

Statistical Analysis: SPSS Version 10.0 (SPSS, Inc.), S-Plus 2000 (Insightful, Inc.), or Prism (GraphPad Software, Inc.) was used for all statistical analyses, all using two-sided tests. The Mann-Whitney U test was used to compare neutralizing antibody responses in the three experimental groups to those in the control group. The independent samples t-test was used to compare log₁₀ peak viral load, log₁₀ set point viral load, and log₁₀ CD4⁺ T cell counts between the experimental and the control groups. Spearman correlation coefficients were used to assess the relationship between pre-challenge neutralizing antibody levels and various markers of disease progression. Finally, Kaplan-Meier survival analysis, the log rank test, and Cox proportional hazards regression were used to compare AIDS-free survival rates in all groups.

### Results

### Neutralization Sensitivity of Glycan Mutants of HIV-1 89.6

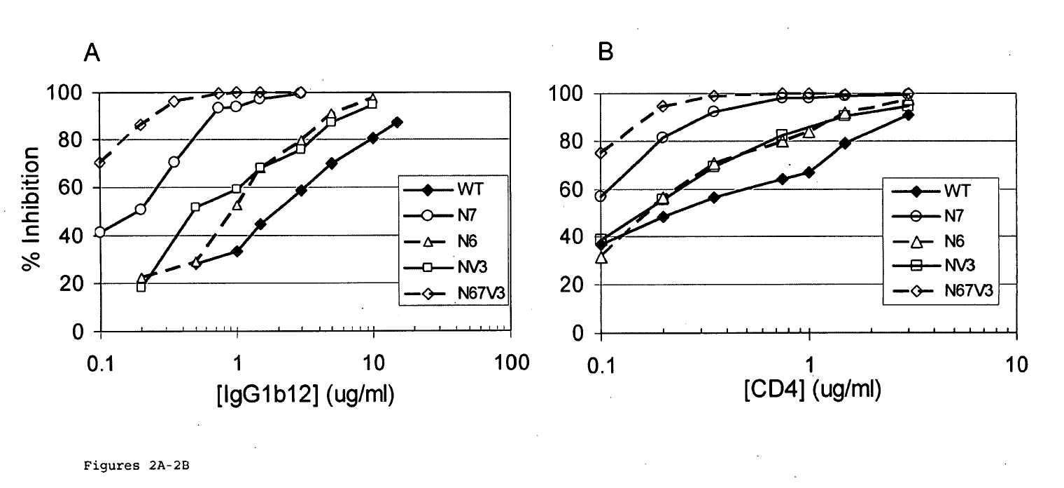

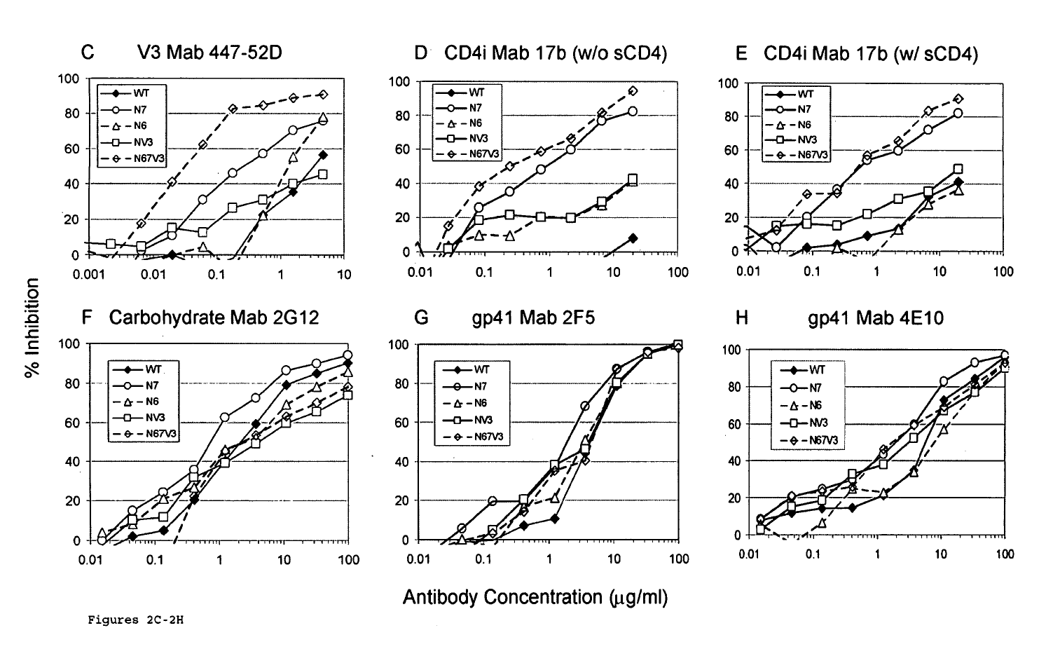

To examine the role of specific glycans in the antigenicity and immunogenicity of HIV-1 Env protein, we introduced mutations substituting the asparagine (N) residue with glutamine (Q) in potential N-linked glycosylation sites (N-X-T/S) in the envelope protein of HIV-1 89.6. Our initial effort included all N-linked glycans in the hypervariable V1, V2 and V3 regions of the surface antigen gp120. For this study, we focused on mutants that meet the following criteria: (1) mutant protein is expressed and processed normally; (2) mutant protein retains overall functional integrity as indicated by successful rescue as viable virus; and (3) mutant virus has enhanced susceptibility to broadly neutralizing antibodies. Three such mutants were identified: N6 (N187Q); N7 (N197Q) and NV3 (N301Q) (30). Compared to the wild-type 89.6, all three mutants showed enhanced sensitivity to a broadly neutralizing monoclonal antibody (MAb) IgG1b12 and to recombinant sCD4-Ig (1) (FIG. 2A-B). Among all mutants tested, N7 showed the most pronounced enhancement in neutralization sensitivity to IgG1b12 (>60-fold) and sCD4-Ig (>50-fold). Combinations of these mutations (N67V3) resulted in even greater sensitivity to these antibodies than single mutants (FIG. 2A-B). We further characterized these mutants (three single mutants and one triple) against a panel of neutralizing monoclonal antibodies. As shown in FIG. 2C, virus pseudotyped with the N7 Env showed increased sensitivity to monoclonal antibodies 447-52D, which recognizes the highly conserved sequence in the tip of the V3 loop. Neither the N6 nor the NV3 mutation affected the susceptibility to 447-52D. However, the triple mutant N67V3 showed even greater sensitivity than N7 to the same antibody, indicating the additive effect of the individual mutations.

Mutants similar to N7 have been introduced in HIV-1 ADA strain by Kolchinsky (61-62) and found to have enhanced sensitivity to a number of neutralizing MAb, including those targeting CD4 binding site, V3, CD4-induced (CD41), and gp41-specific membrane proximal ectodomain region (MPER) epitopes. We therefore examined mutants described here for their sensitivity to CD41 antibody 17b, carbohydrate-dependent antibody 2G12 (52), and MPER antibodies 2F5 and 4E10. As shown in FIG. 2D, all four mutants tested showed increased sensitivity to 17b as compared to WT 89.6 in the absence of sCD4, indicating accessibility of this epitope normally obscured in the unliganded form of gp120. Again, N7 and the triple mutant showed the most pronounced effect. Presence of sCD4 at sub-neutralizing concentrations augmented the neutralizing activities of 17b against the WT 89.6, but had minimal effect on the sensitivity of the glycan mutants (FIG. 2E). Even in the absence of sCD4, both the N7 and the triple mutants were >30-fold more sensitive to 17b as compared to the WT or the N6 and NV3 mutants in the presence of sCD4, indicating the N197 glycan plays a major role in controlling access to the epitope recognized by 17b.

The N7 mutant also showed a modest increase (˜3-fold) in neutralization sensitivity to MAb 2G12 (FIG. 2F), which recognizes a mannan-dependent epitope in gp120. In contrast, the N6 mutant showed little effect and the NV3 mutant showed a modest decrease (2- to 3-fold) in sensitivity to the same antibody, consistent with the differential role of N-linked glycans in modulating the sensitivity to 2G12 (47). Together, the triple mutant N67V3 showed little change from WT in its sensitivity to 2G12, indicating the additive effect of these mutations. None of the mutants tested had any major effect on neutralization by 2F5 (FIG. 2G). However, the N7 and the triple mutants consistently showed ˜3-fold increase in sensitivity to another MPER epitope in gp41, 4E10 (FIG. 2G). The N7 mutant also showed enhanced neutralization sensitivity to polyclonal HIV-1 positive human sera, but was not neutralized by MAb directed to the V1-V2 loop of gp120 from heterologous isolates. Together, these results indicate that all three N-linked glycan we examined can modulate the neutralization sensitivity of HIV-1 89.6. But the major effect was exerted by the N197 glycan located just outside of the carboxy-terminus of the V2 loop.

### CD4-Independent Infection by the N197Q Mutant

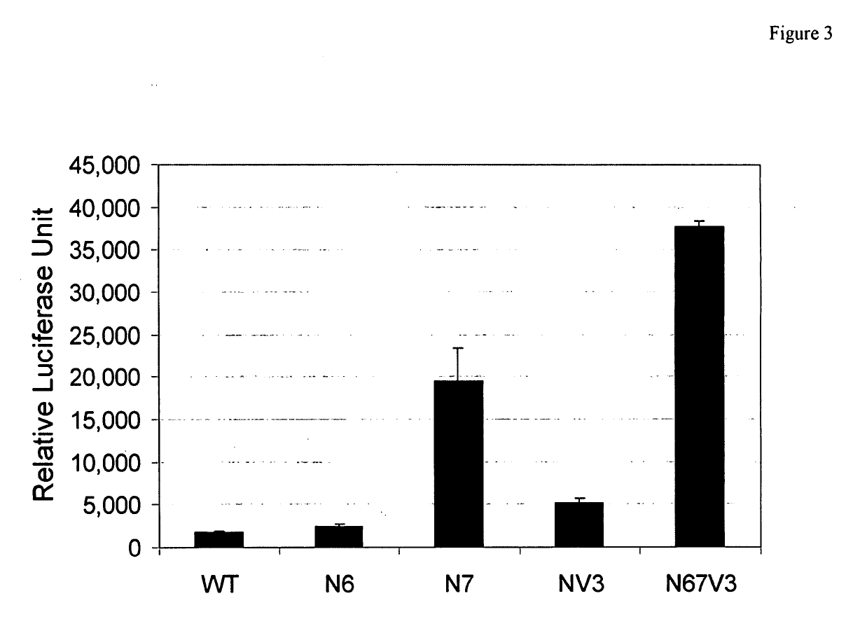

The observation that all three glycan mutant tested showed enhanced neutralization by 17b in the absence of sCD4 suggests that these mutants may have acquired CD4 independent phenotype. This effect was previously reported by Kolchinsky (26) for similar mutants in a CCR5-tropic strain ADA. In this study, we tested all four glycan mutants described for their ability to mediate infection in the absence of CD4. Since the Env of 89.6 uses primarily CXCR4 as the co-receptor, we compared infectivity of HIV-1 pseudotyped with WT or mutant envelopes in U87-CXCR4 cells, using virus inoculum normalized for their infectivity in CD4-expressing U87-CXCR4 cells. Results in FIG. 3 indicate the relative infectivity of the N7 mutant was 10-fold higher than the wr virus in the absence of CD4, confirming the CD4-independent phenotype of the mutant Env. While the N6 mutant inside the V2 loop showed little effect on CD4-dependence, the NV3 mutant consistently showed 2- to 3-fold increase in infectivity in CD4-negative cells as compared to WT Env. Combination of the N7 and the NV3 mutations (e.g. N67V3) resulted in further increase in infectivity in CD4 negative cells. These findings indicate that glycans at both the N7 and, to a lesser extent, the NV3 sites modulate the accessibility to the co-receptor binding site(s) on gp120 and that their effect appears to be additive.

### Immunogenicity of Wild-Type and Mutant Env

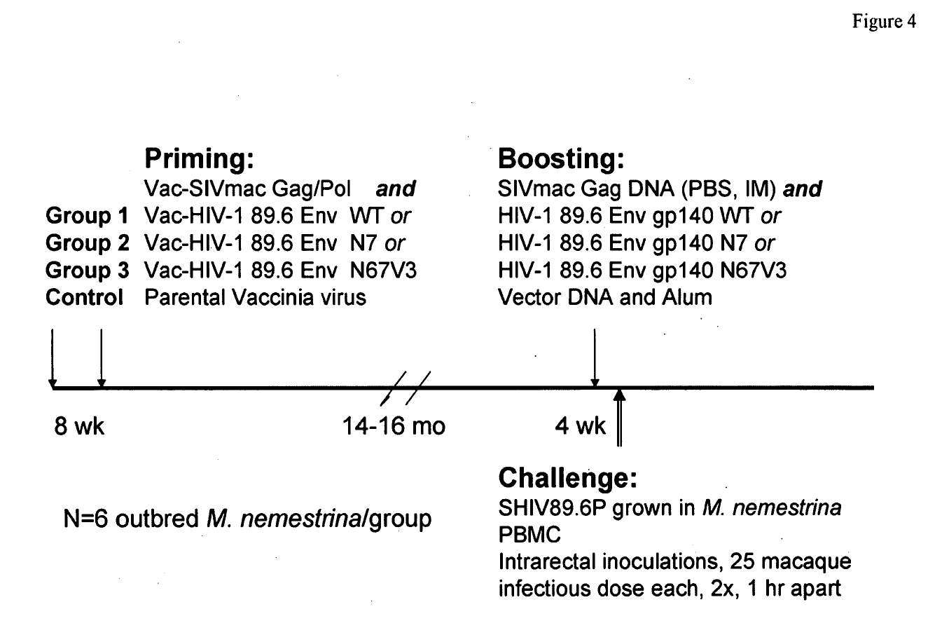

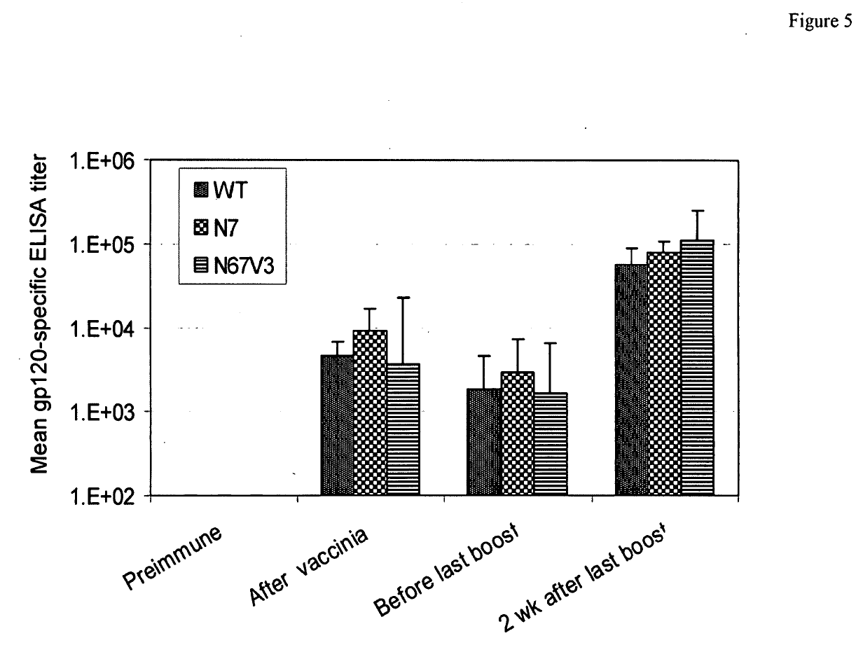

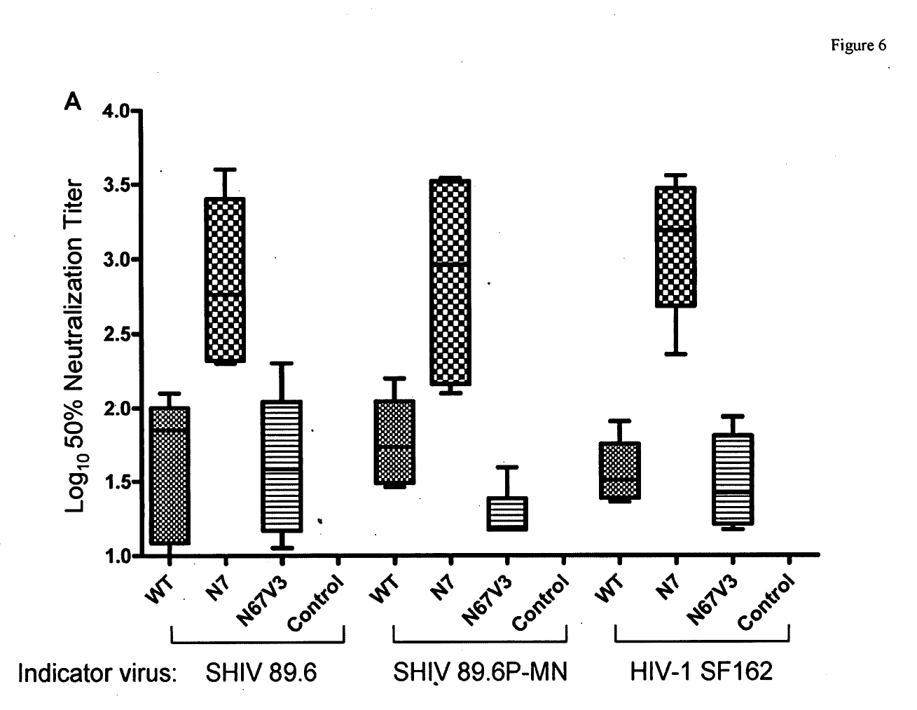

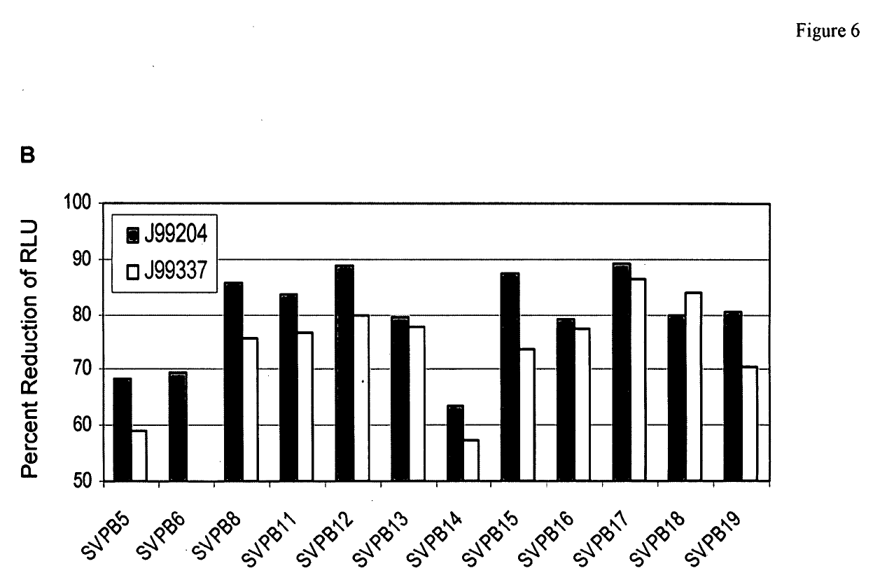

Foregoing observations suggested that removal of one or more N-linked glycans results in the unmasking of several important targets in the envelope protein. These include epitopes for broadly neutralizing antibodies and receptor binding site(s). To test whether such glycan mutants can also affect the immunogenicity of HIV-1 89.6 Env protein, we immunized pig-tailed macaques (N=6/group) with a prime-boost regimen. Since the most pronounced effects in neutralization sensitivity and CD4-independent infection were mediated by the N7 and the triple mutants, we chose to test these two together with WT Env. Animals were inoculated at weeks 0 and 8 with two recombinant vaccinia viruses, one expressing SIVmac239 Gag/Pol, and the other, HIV-1 89.6 Env gp160 in one of three forms: wild-type (Group 1), single mutant N7 (Group 2), or triple mutant containing N6/N7/NV3 (Group 3). Animals were boosted 14 or 16 months later (see Material and Methods) with codon-optimized SIVmac239 gag DNA in saline and the cognate gp140 Env protein formulated in alum (FIG. 4). Control animals were primed with vaccinia virus and boosted with vector plasmid and adjuvant only. Immunization with recombinant vaccinia virus alone elicited low but persistent gp120-specific antibodies. After the final booster immunization, titers increased 30-50-fold to ˜10⁵ in all three groups, indicating a long-lasting immune memory (FIG. 5). However, neutralizing antibody responses differed among the three groups. On day of challenge, Group 1 animals generated only low levels of neutralizing antibodies against SHIV89.6, SHIV89.6P-MN and HIV-1SF162, with median titers of 70, 54, and 32, respectively, whereas Group 2 animals had significantly higher neutralizing activities against the same viruses, with mean titers of 577, 920 and 1563 respectively (FIG. 6A). There was a significant correlation among the 50% neutralization titers against SHIV89.6, SHIV.6P-MN and SF162 (p≦0.0001). Surprisingly, Group 3 animals show only low NtAb against SHIV89.6 and SF162, with little or no activity against SHIV89.6P-MN.

To assess the breadth of response, we tested prechallenge sera from all animals against a standard panel of subtype B primary isolates in a pseudotyped virus neutralization assay as described (30). Viruses selected for this panel have neutralization sensitivity profiles typical of primary subtype B HIV-1 isolates (the so-called Tier 2 isolates). We first tested sera from all animals at 1:15 dilution for their neutralization activity against 7 primary isolates (Table 1). As expected, Group 1 animals immunized with WT Env showed little or no neutralization activity. Only three of the six animals showed low to moderate levels (50%-80% or 80%-90% reduction of infectivity, respectively) of neutralization against three or more of the seven primary isolates tested, including a highly sensitive strain of primary virus (SS1196.1, SVPB9). In contrast, all six animals in Group 2 immunized with mutant N7 Env neutralized one or more primary isolates in addition to SS1196.1. Consistent with results obtained in FIG. 6A, Group 3 animals failed to generate any detectable neutralizing antibody against this primary isolate panel. Of the six control animals, two showed low levels of inhibitory activity in the pseudotyped virus assay. The nature of this activity is not clear. In subsequent studies, we focused on animals J99204 and J99337 from Group 2 (N7 immunized) and evaluated their day-of-challenge sera against pseudotyped viruses representing 12 primary subtype β isolates. Results in FIG. 6B confirmed the breadth of the sera from J99204 and J99337, which neutralized, respectively, 12 of 12 and 11 of 12 isolates tested.

TABLE 1
Neutralization of primary subtype B HIV-1 isolates by day-of-challenge sera
from immunized and control animals:

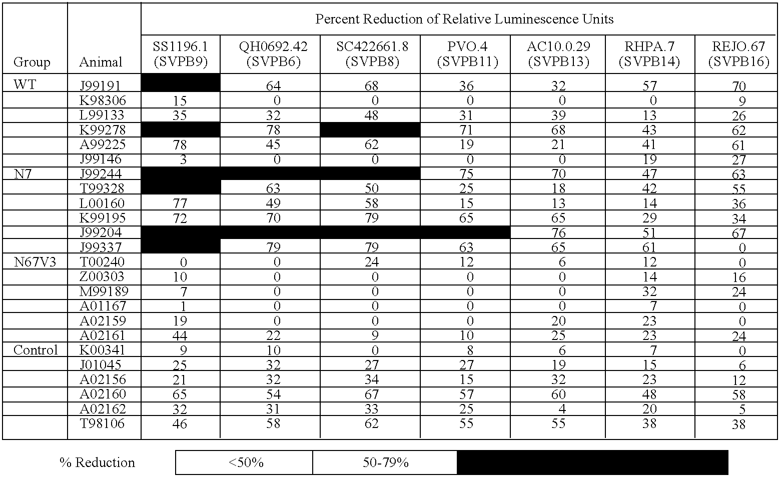

Reduction of Viral Load and Maintenance of Peripheral Blood CD4⁺ T-Cells in Immunized Animals after SHIV89.6P-MN Challenge

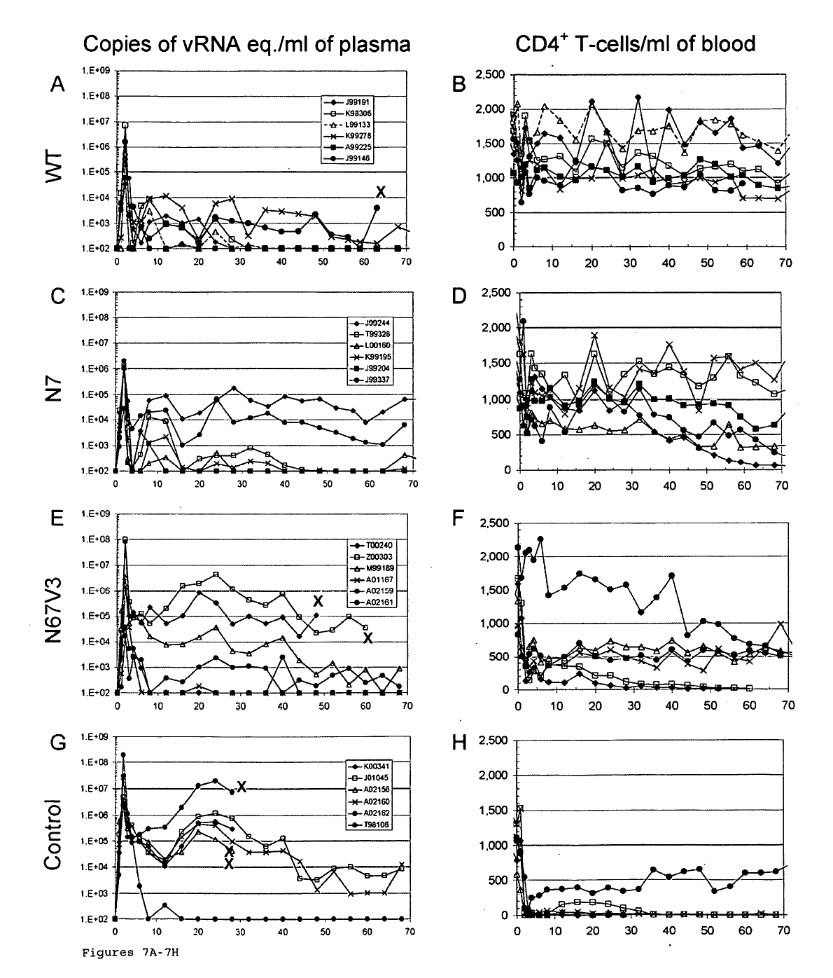

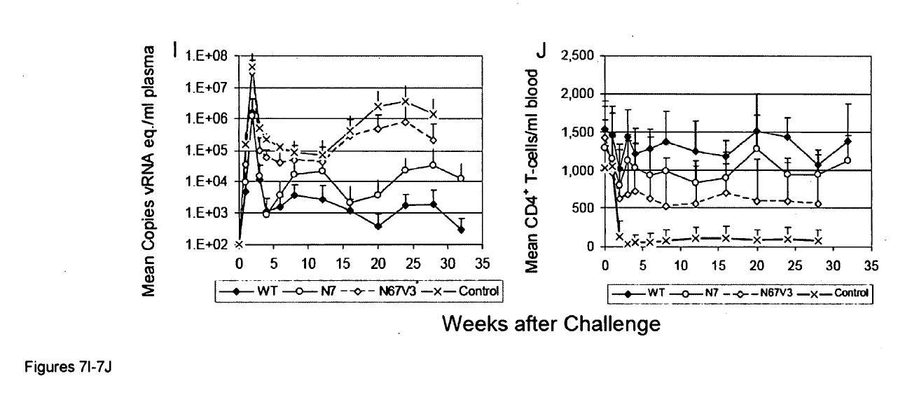

To determine if immune responses elicited by any of the vaccines described could protect macaques against primate lentivirus infection or disease, we challenged all animals four weeks after the last immunization with intrarectal inoculations of a chimeric virus SHIV89.6P-MN. Naïve controls showed high levels of plasma viremia, with a mean peak viral load of 4.4×10⁷ vRNA eq./mL at 2 weeks after infection (FIG. 7, panels G and 1). The mean plasma viral load at week 12 after infection was approximately 10⁵ vRNA eq./ml, increasing to 3×10⁶ vRNA eq./mL by 24 weeks after infection. Animals in Groups 1 and 2 showed >30-fold reduction of peak viral load compared to control animals (p≦0.007 and 0.014, respectively for Groups 1 and 2) (FIG. 7, panels A, C, and 1). Both groups also showed significant (>200-fold) reduction of mean post-acute plasma viral loads (p≦0.01 for both groups compared to controls at week 16). There were no statistically significant differences between Group 1 and 2 animals in their peak and setpoint viral loads after challenge. Although Group 3 animals showed lower mean plasma viral load as compared to the control animals, the difference did not reach statistical significance (p≦0.2). As is expected for SHIV89.6P-MN infection, 5 of the 6 control animals showed rapid and irreversible CD4⁺ T cell loss within a month after challenge. In contrast, all immunized animals, with the exception of two animals (T00240 and Z00303) in Group 3, maintained peripheral blood CD4⁺ T cells within normal range for the first 10 months of the study.

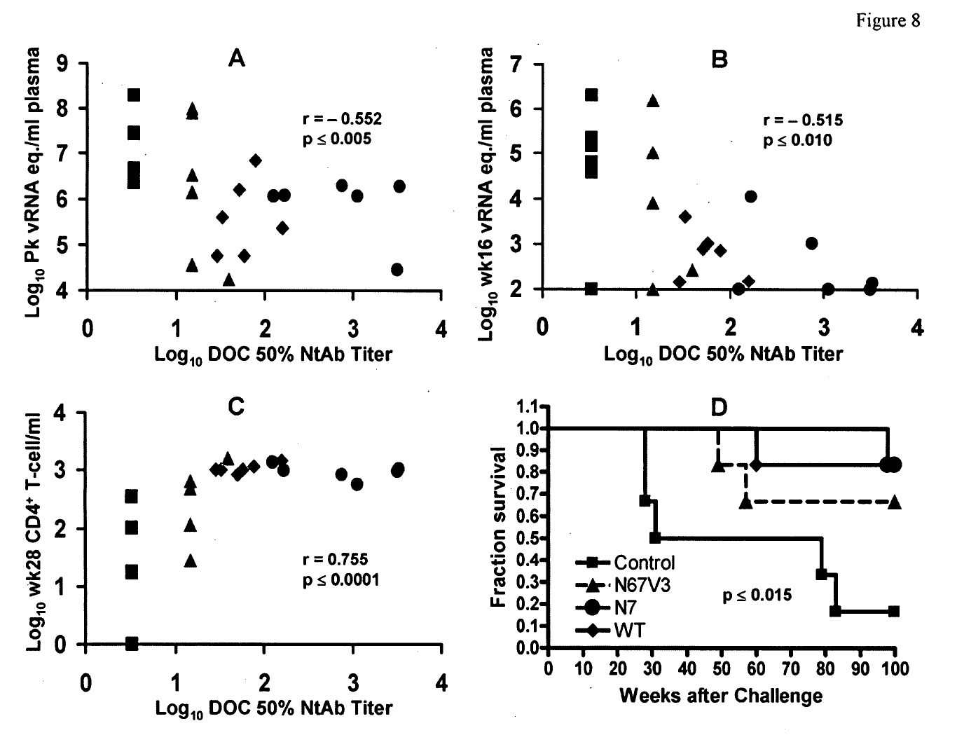

Neutralizing antibody levels from all animals before and after challenge were examined for potential correlates of protective immunity. There is a significant correlation between the neutralization titer against the challenge virus SHIV89.6P-MN on the day of challenge and reduction of peak (wk 2) and setpoint (wk 16) viral loads (p≦0.005 and 0.010, respectively) (FIG. 8A-B). Neutralizing antibody titers against SHIV89.6P-MN on day of challenge were also significantly correlated with the preservation of peripheral blood CD4⁺ T-cells at week 28 (p≦0.0001) (FIG. 8C). Taken together, these results indicate the potential importance of vaccine-induced neutralizing antibodies in the protection or control of infection. As expected from their differences in CD4⁺ T cells, animals in all three immunization groups had significantly greater AIDS-free survival as compared to the unvaccinated controls (p≦0.015). Proportional hazards analysis indicates that animals in Groups 1 and 2 were significantly less likely to die than the control animals (89% and 90% reduction in risk of death for Groups 1 and 2 versus controls, respectively, p-value≦0.04 for both groups). There was a trend for animals in Group 3 to be less likely to die than the control animals (75% reduction in risk), but the difference was not statistically significant (p-value≦0.1).

### Discussion

This example demonstrates that immunization with a mutant Env with a single glycan removed resulted in enhanced NtAb responses, not only against the mutant virus, but also against the homologous wild-type virus and a panel of heterologous primary (“Tier 2”) isolates. This is not predicted by previous findings with glycan-modified envelope proteins. For instance, Bolmstedt et al. (4) compared gp160 and mutant envelope lacking three N-linked glycosylation sites in the V4-V5 region, but only observed preferred homologous NtAb response (i.e., sera from animals immunized with mutant gp160 preferentially neutralized the mutant, but not the wild-type virus). They also immunized mice with DNA lacking the N-terminal V3 (N306) glycan, but failed to detect any difference between the wild-type and the mutant plasmid in their ability to elicit neutralization responses against either the wild-type or the mutant virus . Similarly, Quinones-Kochs et al. (39) reported that neither V1 nor V2 glycan mutant was any better than wild-type HIV-1 89.6 envelope at inducing NtAb. Burke et al. (6) reported improved NtAb response elicited by HIV-1 SF162 Env gp140 with glycan modification at an N-terminal proximal site (residue 154) in V2 loop. However, the breadth of activities detected appears to be limited, neutralizing a heterologous subtype B virus (HIV-1 89.6) only at 1:10 dilution. It should be noted that none of the investigators mentioned above examined the same glycan mutant (N197Q) as we did. The effects of glycan modification on Env immunogenicity therefore appear to be highly dependent on the specific glycans examined.

The basis for the enhanced NtAb response elicited by the N7 mutant remains unclear. As was observed by Kolchinsky et al. (26) with a CCR5-using strain ADA, removal of this V2 loop proximal glycan in the dual-tropic 89.6 strain also enables the mutant envelope to mediate CD4-independent infection. Results reported here further demonstrate that this mutant envelope has increased exposure not only for the coreceptor binding site but also epitopes recognized by several key neutralizing MAb, including those that target the CD4 binding site, the CD4-induced epitope, the tip of the V3 loop and, to a lesser extent, the mannan-dependent epitope defined by 2G12 and the MPER epitope defined by 4E10. Elucidation of the basis of these observations will likely require better understanding of the structure and function of the envelope glycoproteins. However, it is possible that the removal of the N197 glycan results in greater flexibility for the V2 loop, as suggested by Kolchinsky et al. (26). This flexibility could allow the envelope protein to stabilize in a conformation that is normally induced only upon binding to the CD4 receptor during the infection process. As a result, the CD4 binding site, CD4-induced epitope and the V3 loop may become more accessible in the mutant Env. Our observation lends support to approaches that aim to mimic the gp120-CD4 complex, to stabilize the envelope protein in the “liganded form” (28, 53), or to utilize natural isolates that share similar properties as the glycan mutants described here (10, 13, 56, 57).

The triple mutant virus (N67V3) showed greater neutralization sensitivity than the single mutant N7, however, immunization with the triple mutant Env elicited no better NtAb response than the WT protein. Thus, antigenicity does not predict immunogenicity. The basis for the difference is not clear. Virus bearing the triple mutant showed ˜80% reduction of infectivity per microgram of p27 in an infectivity assay when compared with the WT or the other mutant viruses. The expression level and/or the stability of the triple mutant Env also appeared to be reduced compared to the Wr Env in a transient transfection assay. However, these factors are unlikely to contribute to the reduced NtAb responses elicited by the triple mutant Env. First, gp120-specific responses as measured by ELISA (FIG. 5) and Western blot analyses on sera obtained two weeks after the second vaccinia virus immunization showed no difference between the three experimental groups. Second, most of the antigen-specific responses were generated after the booster immunization, when equal quantities of the WT or mutant recombinant gp140 protein were used. Third, gp120-binding antibody titers after the booster immunization were similar among the three immunization groups (FIG. 5). Therefore, it is unlikely that the poor NtAb response elicited in N67V3 immunized animals was due to the lack of expression or overall immunogenicity of the mutant Env protein. Because of the additive effects of the glycan mutations on neutralization sensitivity (FIG. 2) and coreceptor usage (FIG. 3), it is possible that combination of these mutations may have cumulative effects on the overall structure of the Env protein, which in turn may influence its immunogenicity. However, until we have a better understanding of the effect of the glycan modifications on the structure of the Env protein and of the relationship between antigenicity and immunogenicity, it is difficult to predict the outcome of combining different glycan mutations. In this regard, it is noteworthy that previous failures to elicit better NtAb responses with glycan-modified Env proteins often utilized multiply deglycosylated proteins (4, 34, 39). It remains to be determined whether multiple changes may obscure the effects of single mutants (as was observed with the sensitivity to MAb 2G12 when additional glycan mutations were introduced into the N7 mutant). Better understanding of the structural and functional differences between the single and triple glycan mutants described here may provide further insight to the difference in their immunogenicity.

Despite the improved NtAb responses generated by the N7 Env, there were no statistically significant differences in peak and setpoint viral loads between WT- and N7-immunized animals after challenge. Several reasons could account for this apparent discrepancy. First, the level of prechallenge NtAb may still be too low to make a significant difference in the outcome of challenge. This is supported by the finding that none of the vaccines tested here was able to afford protection against infection after challenge with SHIV89.6P-MN. Second, NtAb responses measured in the serum compartment may not accurately reflect responses required to protect against a mucosal challenge. Third, anamnestic response after challenge may minimize the differences in prechallenge vaccine-induced NtAb responses. In support of this notion, animals in the three experimental groups generated similar titers of NtAb against the homologous virus eight weeks after challenge, despite the significant differences between them before challenge. Fourth, it is possible that cellular immunity or humoral responses other than NtAb also contributed to the control of infection. Finally, the lack of a statistically significant difference in viral load in Group 1 and 2 animals despite their differences in prechallenge NtAb responses may simply be due to the small sample sizes. Consistent with this notion is the observation that when data from all the animals in this study were included in the analysis as shown in FIG. 8, there was a significant correlation between the day-of-challenge NtAb titers against the challenge virus and the reduction of viral load (p≦0.005 and 0.010 for peak and setpoint viral loads, respectively) and preservation of CD4⁺ T-cells after challenge (p≦0.0001).

Reduction of viral load and protection against CD4⁺ T cell loss resulting from SHIV89.6P infection has been achieved by a number of vaccines and immunization approaches. Reduction of viral load or “partial protection” was commonly associated with vaccine-elicited CD8⁺ T-cell mediated responses, most notably against the Gag, Pol, Tat and Nef antigens. With few exceptions (31, 42), NtAb against the challenge virus were not elicited by vaccination nor present at the time of challenge, but were induced after challenge and were believed to play a role in the control of infection (15, 16, 18, 40, 41, 45, 55). With the N7 mutant Env, we were able to elicit broadly neutralizing antibody responses, not only against the challenge virus, but also a panel of primary subtype B (“Tier 2”) isolates. Although the potency of the response was insufficient to prevent infection, vaccine-induced NtAb responses are likely to play an important role in the control of infection, since we observed a significant correlation between prechallenge NtAb against the challenge virus and the reduction of viral load and protection against peripheral blood CD4⁺ T-cell loss after SHIV89.6P-MN challenge. It should also be noted that the vaccine was based on 89.6 Env, while the challenge virus was based on the macaque-passaged SHIV89.6P, which most likely contains variants that escape immune responses elicited by SHIV89.6. This observation supports the potential breadth of the protective immunity elicited by the vaccines described.

It is of interest to note that even though animals in Group 3 had little or no NtAb response at the time of challenge, 4 of 6 animals in this group, compared to only 1 of 6 in the control group, were able to recover or maintain normal levels of peripheral blood CD4⁺ T cells after acute infection (FIG. 7F-H). This result indicates a strong trend (p≦0.1) towards a role for vaccine-induced immunity other than prechallenge NtAb in the preservation and recovery of peripheral blood CD4⁺ T cells. As indicated above, cellular immunity and non-neutralizing antibodies (e.g., antibody dependent cellular cytotoxicity) prior to challenge, as well as anamnestic responses following challenge, may have contributed. Furthermore, NtAb present in anatomic sites where virus replicates (e.g. lymph nodes) may also contribute to the control of virus replication in Group 3 animals.

Induction of potent and broadly NtAb against HIV remains a major goal of current AIDS vaccine research. The observation that envelope proteins with a single glycan removed can induce improved NtAb response and control of virus infection indicates a potential role for glycan modification in vaccine design. However, our findings also indicate that the effect may be dependent on the specific glycan modifications studied and that antigenicity per se does not predict immunogenicity.

### REFERENCES CITED IN EXAMPLE 1

1. Allaway, G. P. et al. 1995. AIDS Res. Hum. Retroviruses 11:533-539.
2. Barbas, C. et al. 1992. Proc. Natl. Acad. Sci. U.S. A 89:9339-9343.
3. Barouch, D. H., et al. 2000. Proc. Natl. Acad. Sci. U.S. A 97:4192-4197.
4. Bolmstedt, A., et al. 1996. J. Acquir. Immune. Defic. Syndr. Hum. Retrovirol. 12:213-220.
5. Buchacher, A., et al. 1994. AIDS Res. Hum. Retroviruses 10:359-369.
6. Burke, B., et al. 2006. Virology 355:138-151.
7. Burton, D. R., et al. 1991. Proc. Natl. Acad. Sci. U.S. A 88:10134-10137.
8. Burton, D. R., et al. 1994. Science 266:1024-1027.
9. Chakrabarti, S., et al. 1997. Biotechniques 23:1094-1097.
10. Cham, F., et al. 2006. Virology 347:36-51.
11. Collman, R., et al. 1992. J. Virol. 66:7517-7521.
12. Conley, A. J., et al. 1994. J. Virol. 68:6994-7000.
13. Decker, J. M., et al. 2005. J. Exp. Med. 201:1407-1419.
14. Deng, H., et al. 1996. Nature 381:661-666.
15. Doria-Rose, N. A., et al. 2003. J. Virol. 77:11563-11577.
16. Earl, P. L., et al. 2002. Virology 294:270-281.
17. Egan, M. A., et al. 2000. J. Virol. 74:7485-7495.
18. Egan, M. A., et al. 2004. AIDS Res. Hum. Retroviruses. 20:989-1004.
19. Gorny, M. K., et al. 1992. J. Virol. 66:7538-7542.
20. Gorny, M. K., et al. 1997. J. Immunol. 159:5114-5122.
21. Gorny, M. K., et al. 1993. J. Immunol. 150:635-643.
22. Hu, S.-L., et al. 1986. Nature 320:537-540.
23. Hu, S.-L., et al. 1990. Virology 179:321-329.
24. Hu, S.-L., et al. 1992. AIDS Res. Hum. Retroviruses. 8:1489-1494.
25. Klaniecki, J., et al. 1991. AIDS Res Hum Retroviruses 7:791-798.
26. Kolchinsky, P., et al. 2001. J. Virol. 75:3435-3443.
27. Kolchinsky, P., et al. J. Virol. 75:2041-2050.
28. Kwong, P. D., et al. 1998. Nature 393:648-659.
29. Li, M., et al. 2005. J. Virol. 79:10108-10125.
30. Li, Y., et al. 2001. AIDS Res. Hum. Retroviruses 17:1473-1479.
31. Mascola, J. R., et al. 2005. J. Virol. 79:771-779.
32. McCaffrey, R. A., et al. 2004. J. Virol. 78:3279-3295.
33. Montefiori, D. C. 2004. p. 12.11.1-12.11.15. In J. E. Coligan, et al (eds.), Current Protocols in Immunology. John Wiley & Sons.
34. Mori, K., et al. 2005. J. Virol. 79:10386-10396.
35. Nyambi, P. N., et al. 1998. J. Virol. 72:9384-9391.
36. Polacino, P. S., et al. 1999. J. Virol. 73:8201-8215.
37. Purtscher, M., et al. 1996. AIDS 10:587-593.
38. Purtscher, M., et al. 1994. AIDS Res. Hum. Retroviruses. 10:1651-1658.
39. Quinones-Kochs, M. I., et al. 2002. J. Virol. 76:4199-4211.
40. Ramsburg, E., et al. 2004. J. Virol. 78:3930-3940.
41. Rasmussen, R. A., et al. 2002a. J. Med. Primatol. 31:40-60.
42. Rasmussen, R. A., et al. 2002b. AIDS 16:829-838.
43. Reimann, K. A., et al. 1996. J. Virol. 70:3198-3206.
44. Roben, P., et al. 1994. J. Virol. 68:4821-4828.
45. Rose, N. F., et al. 2001. Cell 106:539-549.
46. Salter, R. D., et al. 1985. Immunogenetics 21:235-246.
47. Sanders, R. W., et al. 2002 J. Virol. 76:7293-7305.
48. Sullivan, N., et al. 1998. J. Virol. 72:4694-4703.
49. Suryanarayana, K., et al. 1998. AIDS Res. Hum. Retroviruses 14:183-189.
50. Thali, M., et al. 1993. J. Virol. 67:3978-3988.
51. Trkola, A., et al. 1996a. Nature 384:184-187.
52. Trkola, A., et al. 1996b. J. Virol. 70:1100-1108.
53. Wyatt, R., et al. 1998. Nature 393:705-711.
54. Wyatt, R., et al. 1995. J. Virol. 69:5723-5733.
55. Xu, R., et al. 2006. Virology 349:276-289.
56. Zhang, P. F., et al. 2002. J. Virol. 76:644-655.
57. Zhang P. F., et al. 2007. Proc. Natl. Acad. Sci. U.S.A. 104:10193-10198.
58. Zolla-Pazner, S., et al. 1995. J. Virol. 69:3807-3815.

### Example 2

### Sequential Immunization to Preferentially Expand Antibodies to Common Epitomes

This example demonstrates an optimization strategy for a sequential immunization approach designed to preferentially expand antibodies to common epitopes (referred to as “PEACE”). Improvements in immunization regimen, including the use of more potent adjuvants and repeated boosting with CD4-independent mutant Env from different isolates, are designed to result in greater potency and/or breadth in neutralizing antibody responses, which contribute to the protection against HIV-1 infection and diseases.

The results of Example 1 above indicate that immunization with mutant Env with more exposed conserved epitopes can enhance the breadth of NtAb responses. While vaccinia virus prime and protein boost is an effective approach to generate antibody responses, in the above example, we only used a single booster immunization with proteins formulated in alum. This example provides guidance for two interrelated approaches: (1) to test the effect of repeated boosting with Env formulated in more potent, but commercially available adjuvants; and (2) to test a new concept of preferential expansion of antibodies to common epitopes (PEACE) by repeated boosting with heterologous Env, all exhibiting CD4 independent phenotype as the common feature. PEACE is an extension of the heterologous “prime-boost” concept we and others developed. That is, by priming and boosting with different vaccines presenting a common immunogen, we can expand responses to shared epitope(s), without boosting those that are unique to each individual component (e.g., vector-specific responses in virus vector-protein prime-boost regimens). Many variations on the original prime-boost theme have been explored over the years, including DNA-vector immunization to enhance cellular responses and sequential boosting with related Env vaccines.

This approach can enhance the breadth of response by at least two mechanisms: (1) preferentially expanding responses to common epitopes (in this case, epitopes exposed as a result of the acquisition of CD4-independent phenotype), and (2) increasing the diversity of responses directed to less conserved, or “type-specific” neutralizing epitopes on different Env. The breadth of protective efficacy can be evaluated by using homologous as well as heterologous SHIV challenge models.

As indicated in Example 1, the enhanced response elicited by the N7 mutant Env was achieved with only one booster immunization with the protein formulated in alum. One can increase the potency and the breadth of response by increasing the number of booster immunizations and incorporating the use of more potent adjuvants. Several commercially available adjuvants commonly used for animal studies include the RIBI adjuvant, Titermax, and incomplete Freund's adjuvant (IFA). Four groups of rabbits, 6 per group, all primed with the same recombinant vaccinia virus expressing the N7 mutant Env at weeks 0 and 8 and are then boosted with the oligomeric N7 Env gp140 formulated in one of the three adjuvants. In addition, alum can be used as a comparator. Booster immunization is repeated every 3-4 months when antibody titer decay has stabilized.

When homologous NtAb is observed, the sera is then tested against two heterologous primary subtype β isolates, one that is neutralized by existing sera from N7 immunized animals (e.g., SC422661.8) and one that is not (e.g., RHPA.7). A total of six booster immunizations with the same protein immunogen and adjuvant are administered. This number of immunizations (total of 8, including the priming with recombinant vaccinia virus) is comparable to that used by Haigwood et al (1992, J. Virology 66:172-182) and by Grundner et al., (2005), both of whom observed low level of cross-subtype neutralizing antibodies by repeated boosting with subunit Env proteins. The boosting can be stopped sooner if untoward effects are observed due to immunization. Animals are monitored for general health and bled before and 2 weeks after each immunization for serological studies.

Sera collected at the timepoint with the highest neutralizing activities in the initial screening test is further analyzed for the potency and breadth of response, first against a full panel of subtype B tier 2 isolates and then against subtype A and C isolates (tier 3). One can perform binding (ELISA) competition assays as described by Richardson et al. (1996) with homologous V3 and gp41 peptides and V3 fusion proteins as competitors. The ability of purified IgG from immunized animals to compete with the binding of biotinylated Mab (IgG1b12, 2G12, 447D, 17b, 2F5 and 4E10) to homologous gp140 can also be examined. Lastly, neutralization competition assays can be performed with 89.6 WT, N7, and SF162 Env pseudotyped virus as the indicator, and homologous V3 peptide, V3 fusion protein, or gp41 peptides as the competitor. Results from these studies will inform (1) the extent to which repeating boosting will increase the breadth and potency of the NtAb response; (2) the extent to which the use of adjuvants other than alum will increase the potency of NtAb response, or the number of immunizations needed to achieve this response; and (3) whether any change in the breadth or potency of the NtAb response can be correlated with changes in the epitope specificity of the response.

The implementation of PEACE builds upon experiments described above and addresses the further enhancement of cross-reactive NtAb responses by boosting with different Env immunogens that share a common phenotype, that is, the ability to mediate CD4-independent infection.

Animals are primed with recombinant vaccinia virus expressing Env gp160 and boosted with oligomeric Env gp140. Based on studies described earlier in this example, one can select the adjuvant and the number of booster immunizations to achieve optimal response. The WT and CD4-independent mutant Env are used for the primary immunization and the first booster immunization (Table 2, Groups 1 and 2, below).

To determine if there is any difference between priming with a subtype B vs. a subtype C Env, one can include Groups 3 and 4 (Table 2), which will be primed with subtype C Env, both the WT and the best CD4-independent mutants identified. We have previously constructed recombinant vaccinia virus and produced subunit Env protein from another subtype C isolate, 1084i (Zhang et al., 2006, Retrovirology 3:73; Rasmussen et al., 2006, Vaccine 24:2324-2332), which can be used as the second WT subtype C (WT C2) immunogen for this study. The experimental design and immunization regimen is summarized below:

TABLE 2
Repeated immunization regimen for PEACE

| Group | Prime (Vac-gp160) | Boost 1 | Boost 2 | Boost 3 | Boost 4 | Boost 5 | Boost 6 |
|---|---|---|---|---|---|---|---|
| 1 | WT Env B1 | WT B1* | WT B2 | WT B3 | WT B4 | WT C1 | WT C2 |
| 2 | Mutant Env B1 | Mut B1 | Mut B2 | Mut B3 | Mut B4 | Mut C1 | Mut C2 |
| 3 | WT Env C1 | WT C1 | WT C2 | WT B1 | WT B2 | WT B3 | WT B4 |
| 4 | Mutant Env C1 | Mut C1 | Mut C2 | Mut B1 | Mut B2 | Mut B3 | Mut B4 |

*B1-B4 refer to the four subtype B isolates from which CD4-independent mutants are used.

These include HXBc2, ADA, SF162 and 89.6 PM-SI. WT and Mut refer to the WT and mutant Env, respectively. Two subtype C mutant Env, Mut C1 and Mut C2, are selected from rabbit studies, based on the potency and breadth of NtAb they induce.

Sera are collected before and after each immunization as before. The breadth of NtAb response is evaluated against standard panels of subtype B or C primary isolates. Animals that receive sequential immunizations with different WT Env will exhibit a broadening of response, but this broadening is more pronounced and the NtAb more potent in animals that receive different CD-independent mutant Env in similar immunization regimens. Because subtype C Env is able to engender cross-reactive NtAb, one can expect to see the most cross-reactive responses in Group 4 above.

Because Gag-specific responses contribute to protection and because most if not all current SHIV models share the same SIVmac239 backbone, the inclusion of SIV Gag in the immunogen may complicate the interpretation of results. Therefore only Env is included as the vaccine immunogen in this study. Thirty-six pig-tailed macaques are used. Twelve animals in each experimental arm are primed with recombinant vaccinia virus expressing either the WT or the CD4-independent Env gp160 and boosted with the cognate oligomeric Env gp140 protein. The remaining 12 animals are primed with parental vaccinia virus and boosted with adjuvant only. Four weeks after the last immunization, all animals are challenged with an intrarectal inoculation of SHIV (at a predetermined inoculum containing 2-10 50% animal infectious doses). Half of the animals (6 each from the WT, or the mutant Env immunized arms and 6 from the control arms) are challenged with SHIV expressing a homologous Env and the other with a heterologous one. The choice of SHIV will depend on the results obtained in the rabbit studies. If the NtAb elicited are limited in breadth, one can use homologous and heterologous SHIV representing the same subtype (e.g., SHIV162P and SHIV89.6P). If cross-subtype NtAb is elicited, one can use two heterologous SHIV challenges, one from subtype B (e.g. SHIV162P4) and the other subtype C. A robust subtype C SHIV (SHIV 1157ipd3N4) challenge model has recently been developed in the laboratory of Dr. Ruprecht (Song et al., 2006, J. Virol. 80:8729-8738).

Macaques are bled before and after each immunization. Gp120-specific ELISA is used to monitor binding antibody responses. Sera with the highest ELISA titers (most likely after the last booster immunization) are first tested for NtAb activities against pseudotype viruses bearing the WT and mutant Env of the vaccine strains. Breadth of response is then tested against standard panels of subtype B and C primary isolates at a single dilution (1:15), using preimmune sera from each animal as the baseline. Presence of CD4-induced NtAb is examined. After challenge inoculation, animals are monitored for virologic, immunologic and clinical responses.

### Example 3

### Effect of N197 Glycan Mutations on the Neutralization Sensitivity of a Clade A HIV-1 Isolate

This example demonstrates that removal of the same N197N-linked glycan described above resulted in enhanced exposure of the CD4 receptor binding site. These results confirm the observation described above with a clade B virus envelope, supporting the applicability of the vaccine described herein to non-clade B viruses.

Env clones were obtained from a Clade A HIV-1 isolate from an individual with unusually broad neutralizing antibody (NtAb) responses. Mutant Env clones were constructed with a serine (199S) or an alanine (199A) at amino acid 199, which forms a part of the potential N-linked glycan (PNLG) sequon (N-X-SIT) at N197. Both Env mutants were successfully rescued as infectious pseudotyped viruses, using methods as described by Long et al. (AIDS Res Hum Retroviruses 18: 567-576, 2002). Infectivity of the pseudotyped viruses was determined as 50% tissue culture infectious dose (TCID₅₀) in TZM-bl cells. Neutralization was measured by reduction of luciferase gene expression as previously described (Montefiori, p. 12.11.1-12.11.15, in J. E. Coligan et al. (eds.), Current Protocols in Immunology, John Wiley & Sons, 2004). Briefly, indicator viruses containing 200 TCID₅₀ was incubated with different concentrations of the test antibody in triplicates in a total volume of 150 μL for 1 hr at 37° C. in 96-well flat-bottom culture plates. Freshly trypsinized cells (10,000 cells in 100 μl of growth medium containing 75 μg/mL DEAE dextran) were added to each well. One set of control wells received cells+virus (virus control) and another set received cells only (background control). After a 48-hour incubation, 100 μL of cells were transferred to a 96-well black solid plates (Costar) for assays of luciferase activities using Bright Glo substrate solution as described by the supplier (Promega). Neutralization was expressed as % reduction of infectivity measured as relative luminescence units.

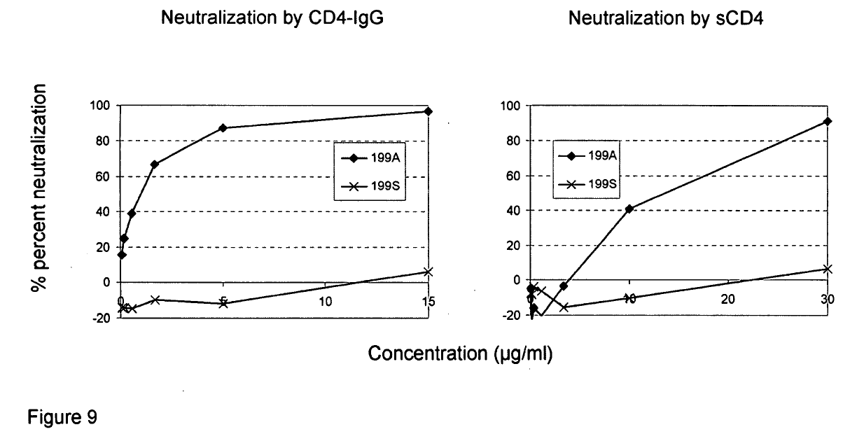

As shown by data presented in FIG. 9, the 199S virus (with the PNLG site present) was highly resistant to neutralization by soluble CD4 (sCD4) and CD4-IgG fusion protein. Mutation at this PNLG significantly increased its neutralization sensitivity, indicating increase accessibility or exposure of the CD4 binding site. This finding supports that notion that the effect observed (Example 1 above and Li et al., J. Virol. 82: 638-651, 2008) in a dade B virus, 89.6, is not isolate-specific, but is shared by HIV-1 from a different clade.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A pharmaceutical composition comprising a polynucleotide encoding an envelope protein comprising gp120 of human immunodeficiency virus-1 (HIV-1), and a pharmaceutically acceptable carrier, wherein the gp120 has a mutation at amino acid residues 197-199, and wherein the mutation effects removal of a single N-linked glycan relative to wild type gp120.

2. The composition of claim 1, wherein the mutation is at N197.

3. The composition of claim 1, wherein the mutation is at T/S199.

4. The composition of claim 1, wherein the envelope protein comprises gp140 or gp160.

5. The composition of claim 1, wherein the mutation is N197Q.

6. The composition of claim 1, wherein the gp120 has the amino acid sequence of SEQ ID NO: 5.

7. The composition of claim 1, wherein the HIV-1 is isolate 89.6.

8. The composition of claim 1, further comprising an adjuvant.

9. A pharmaceutical composition comprising a recombinant virus genetically modified to express an envelope protein comprising gp120 of human immunodeficiency virus-1 (HIV-1), wherein the gp120 has a mutation at amino acid residues 197-199, and wherein the mutation effects removal of a single N-linked glycan relative to wild type gp120.

10. The composition of claim 9, wherein the virus is an adenovirus, adeno-associated virus, pox virus, or alphavirus.

11. The composition of claim 10, wherein the pox virus is a vaccinia virus.

12. The composition of claim 9, wherein the virus is an attenuated HIV virus expressing the mutant gp120.

13. A pharmaceutical composition comprising gp120 of human immunodeficiency virus-1 (HIV-1), wherein the gp120 has a mutation at amino acid residues 197-199, and wherein the mutation effects removal of a single N-linked glycan relative to wild type gp120.

14. The composition of claim 13, wherein the mutation is N197Q.

15. A method of producing immune cells directed against HIV comprising contacting an immune cell with an antigen-presenting cell, wherein the antigen-presenting cell is modified to present an envelope protein comprising gp120 of human immunodeficiency virus-1 (HIV-1), wherein the gp120 has a mutation at amino acid residues 197-199, and wherein the mutation effects removal of a single N-linked glycan relative to wild type gp120.

16. A method of reducing plasma levels of HIV in a subject, comprising immunizing the subject with the composition of claim 1.

17. A method of maintaining CD4 cells in an HIV-infected subject, comprising administering the composition of claim 1 to the subject.

18. A method of prolonging disease-free survival in an HIV-infected subject, comprising administering the composition of claim 1 to the subject.

19. A method of enhancing production of HIV-neutralizing antibody in a subject comprising contacting an HIV-infected cell in a subject with the composition of claim 1.

20. A method of inducing an immune response to HIV or HIV infected cells in a subject comprising administering the composition of claim 1 to the subject.

21. A method of treating an HIV infection in a subject comprising administering the composition of claim 1 to the subject.

22. The method of claim 21, further comprising administering the composition a second time after at least 3 weeks following the previous administration.


SEQUENCE LISTING (17 sequences)

SEQ ID NO: 1 - protein, 44 aa, Human immunodeficiency virus
CSFYITTSIRNKVKKEYALFNRLDVVPIENTNNTKYRLISCNTS

SEQ ID NO: 2 - protein, 35 aa, Human immunodeficiency virus
CTRPNNNTRRRLSIGPGRAFYARRNIIGDIRQAHC

SEQ ID NO: 3 - protein, 853 aa, Human immunodeficiency virus
MRVKEIRKNWQHLRGGILLLGMLMICSAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCQTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRAVGIGAVFLGFLGAAGSTMGAASVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMEWEREIDNYTDYIYDLLEKSQTQQEKNEKELLELDKWASLWNWFDITNWLWYIRLFIMIVGGLIGLRIVFAVLSIVNRVRQGYSPLSFQTLLPASRGPDRPEGTEEEGGERDRDRSGPLVNGFLALFWVDLRNLCLFLYHLLRNLLLIVTRIVELLGRRGWEALKYWWNLLQYWSQELKNSAVSLLNATAIAVAEGTDRVIKIVQRACRAIRNIPTRIRQGLERALL

SEQ ID NO: 4 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 5 - protein, 508 aa, Human immunodeficiency virus
MRVKEIRKNWQHLRGGILLLGMLMICSAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCQTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKR

SEQ ID NO: 6 - DNA, 2564 bp, Human immunodeficiency virus
caatgagagtgaaggagatcaggaagaattggcagcacttgagagggggcatcttgctccttgggatgttgatgatctgtagtgctgcaaaagaaaaaacgtgggtcacaatctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaatttttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagcagtgggaataggagctgtgttccttgggttcttgggagcagcaggaagcactatgggcgcagcgtcagtgacgctgacggtacaggccaggctattattgtctggtatagtgcagcagcagaacaatctgctgagggctattgaggcgcaacagcatatgttgcaactcacagtctggggcatcaagcagctccaggcaagagtcctggctctggaaagatacctaagggatcaacagctcatgggaatttggggttgctctggaaaactcatttgcaccacttctgtgccttggaatgttagttggagtaataaatctgtggatgatatttggaataacatgacctggatggagtgggaaagagaaattgacaattacacagactatatatatgacttacttgaaaaatcgcaaacccaacaagaaaagaatgaaaaagaattattggaattggataaatgggcaagtttgtggaattggtttgacataacaaactggctgtggtatataagattattcataatgatagtaggaggcttgataggtttaagaatagtttttgctgtactttctatagtaaatagagttaggcagggatattcaccattatcgtttcagaccctcctcccagcctcgaggggacccgacaggcccgaaggaacagaagaagaaggtggagagagagacagagacagatccggtccattagtgaacggattcttggcacttttctgggtcgatttgaggaacctgtgcctcttcctctaccacctcttgagaaacttactcttgattgtaacgaggattgtggaacttctgggacgcagggggtgggaagccctcaaatattggtggaatctcctgcaatattggagccaggaactaaagaatagtgctgttagcttgctcaacgccacagccatagcagtagctgaggggacagatagggttataaaaatagtacaaagagcttgtagagctattcgcaacatacctacaagaatcagacagggcttggaaagggctttgctataa

SEQ ID NO: 7 - DNA, 1526 bp, Human immunodeficiency virus
caatgagagtgaaggagatcaggaagaattggcagcacttgagagggggcatcttgctccttgggatgttgatgatctgtagtgctgcaaaagaaaaaacgtgggtcacaatctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaatttttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaaga

SEQ ID NO: 8 - DNA, 1335 bp, Human immunodeficiency virus
ataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagcagtgggaataggagctgtgttccttgggttcttgggagcagcaggaagcactatgggcgcagcgtcagtgacgctgacggtacaggccaggctattattgtctggtatagtgcagcagcagaacaatctgctgagggctattgaggcgcaacagcatatgttgcaactcacagtctggggcatcaagcagctccaggcaagagtcctggctctggaaagatacctaagggatcaacagctcatgggaatttggggttgctctggaaaactcatttgcaccacttctgtgccttggaatgttagttggagtaataaatctgtggatgatatttggaataacatgacctggatggagtgggaaagagaaattgacaattacacagactatatatatgacttacttgaaaaatcgcaaacccaacaagaaaagaatgaaaaagaattattggaattggataaatgggcaagtttgtggaattggtttgacataacaaactggctgtggtatataagattattcataatgatagtaggaggcttgataggtttaagaatagtttttgctgtactttctatagtaaatagagttaggcagggatattcaccattatcgtttcagaccctcctcccagcctcgaggggacccgacaggcccgaaggaacagaagaagaaggtggagagagagacagagacagatccggtccattagtgaacggattcttggcacttttctgggtcgatttgaggaacctgtgcctcttcctctaccacctcttgagaaacttactcttgattgtaacgaggattgtggaacttctgggacgcagggggtgggaagccctcaaatattggtggaatctcctgcaatattggagccaggaactaaagaatagtgctgttagcttgctcaacgccacagccatagcagtagctgaggggacagatagggttataaaaatagtacaaagagcttgtagagctattcgcaacatacctacaagaatcagacagggcttggaaagggctttgcta

SEQ ID NO: 9 - DNA, 39 bp, Artificial sequence
  primer
gtagtaccaatagaaaatactcagaatactaagtatagg

SEQ ID NO: 10 - DNA, 39 bp, Artificial sequence
  primer
cctatacttagtattctgagtattttctattggtactac

SEQ ID NO: 11 - DNA, 38 bp, Artificial sequence
  primer
ggttaataagttgtcagacctcagtcattacacaggcc

SEQ ID NO: 12 - DNA, 38 bp, Artificial sequence
  primer
ggcctgtgtaatgactgaggtctgacaacttattaacc

SEQ ID NO: 13 - DNA, 37 bp, Artificial sequence
  primer
gtacaagacccaaccagaatacaagaagaaggttatc

SEQ ID NO: 14 - DNA, 37 bp, Artificial sequence
  primer
gataaccttcttcttgtattctggttgggtcttgtac

SEQ ID NO: 15 - DNA, 25 bp, Artificial sequence
  primer
actttcggtcttagctccattagtg

SEQ ID NO: 16 - DNA, 23 bp, Artificial Sequence
  primer
ttttgcttcctcagtgtgtttca

SEQ ID NO: 17 - DNA, 28 bp, Artificial sequence
  primer
ttctcttctgcgtgaatgcaccagatga